(19) United States
(12) Patent Application Publication
(10) Pub. No.: US 2003/0215529 A1
(43) Pub. Date: Nov. 20, 2003

(54) Composition and pharmaceutical preparation containing same for the treatment of herpes and related viral infections

(75) Inventors: Song F. Lee, Halifax (CA); Paul P.H. But, N.T. (HK); Spencer H.S. Lee, Halifax (CA); Vincent E.C. Ooi, N.T. (HK); Hong-Xi Xu, Halifax (CA); Yongwen Zhang, Shatin (CN); Wai-Chong Foong, Bedford (CA); Jonathan Blay, Bedford (CA)

Correspondence Address: FOLEY & LARDNER, P.O. BOX 80278, SAN DIEGO, CA, 92138-0278, US

(73) Assignees: Dalhousie University; The Chinese University of Hong Kong

(21) Appl. No.: 10/265,354

(22) Filed: Oct. 4, 2002

Related U.S. Application Data
(63) Continuation-in-part of application No. 09/160,210, filed on Sep. 23, 1998, now abandoned.

(51) Int. Cl. A61K 35/78

(52) U.S. Cl. 424/725; 514/22

(57) ABSTRACT

In accordance with the present invention, novel compositions useful for the treatment of the cytopathogenic effects of an enveloped virus in mammals have been discovered by extraction and purification from the spikes of Prunella vulgaris. In particular, invention compositions comprise a lignin-carbohydrate complex as an active ingredient for inhibition of viral infection in a mammal. In accordance with an embodiment of the present invention, it has been discovered that invention compositions are effective agents for the prophylaxis and therapy in mammals of diseases caused by enveloped viruses, e.g., herpes simplex virus. Methods for producing invention compositions and uses therefor are also provided.

### RELATED APPLICATIONS

This application is a continuation-in-part of U.S. patent application Ser. No. 09/160,210, filed Sep. 23, 1998, now pending, which claims priority from U.S. Provisional Application No. 60/059,775, filed Sep. 23, 1997, both incorporated by reference herein in their entirety.

### FIELD OF THE INVENTION

The present invention relates to a novel composition which can be extracted and purified from the spikes of Prunella vulgaris. Invention composition is an effective agent for treatment of the cytopathogenic effects of an enveloped virus in mammals. In another aspect, the present invention relates to methods for the treatment of the cytopathogenic effects of an enveloped virus and related indications in mammals, employing the invention composition as the active agent. In yet another aspect, the present invention relates to methods for the treatment of the cytopathogenic effects of an enveloped virus in mammals exposed to immunosuppressive regimens. In still another aspect, the present invention relates to methods for protecting mammals from reactivated viral infection during or following treatment with chemotherapeutic agents. In a further aspect, the present invention relates to methods for obtaining invention compositions having antiviral action from Prunella vulgaris, and formulations containing said compositions. In a still further aspect, the present invention relates to methods of purification of invention compositions from Prunella vulgaris and characterization thereof. In a particular aspect, the invention relates to compositions comprising lignin-carbohydrate complexes having anti-HSV activities.

### BACKGROUND OF THE INVENTION

Viral infections caused by enveloped viruses are a heterogenous group of disorders characterized by viral infection of host cells. An example of such infections includes acquired herpes virus infections caused by broad categories of herpes-related viruses, including the alpha-, beta- and gamma-herpes viruses, and where the onset of infection is characterized by herpes simplex virus (HSV) infection of host cells. Primary herpes virus infections are normally acquired in childhood, but later enter a dormant phase (e.g., in the nerves). Reactivation of herpes virus infections result from a variety of factors, such as ultraviolet light, stress, and adult onset.

Extensive work is being done to identify compounds that inhibit the pathogenicity of viruses to prevent or treat viral infections. While some anti-viral agents are presently available, there is a great need for additional compounds that would be more active, and more specific in preventing or treating various viral infections.

The incidence and severity of herpes virus infections have increased over the last decade as a result of disease pattern, the frequency of drug use and the emergence of drug and analog resistant herpes infections, resulting in new interest in the pathophysiology of HSV enveloped virus diseases and their progression to identify more active and more specific compounds to prevent or treat HSV infections.

Infection with HSV-1 is a problem worldwide, regardless of race and geography. Approximately 70% of people older than 40 have antibodies against HSV-1 (Rawis et al., Elsevier (North-Holland) Amsterdam, 137-152, 1981). Approximately 16-35%, 40-80%, and over 90% of the United States population are seropositive for or infected by herpes simplex virus type 1 (HSV-1), herpes simplex virus type 2 (HSV-2), and varicella zoster virus (VZV), respectively. HSV-1 is the cause of cold-sores, keratitis, and encephalitis. HSV-2 is responsible for genital herpes and VZV is the causative agent for chicken pox and shingles. Five other members of the herpes family, specifically the Epstein-Barr virus (infectious mononucleosis and Burkitt's lymphoma), cytomegalovirus (congenital CMV infections), a human herpes virus 6 and 7 (fever and rash in children), and human herpes virus 8 (Kaposi's Sarcoma), also cause disease in humans. In serious cases, HSV may result in encephalitis with severe neurological sequelae and death (Kastrukoff et al., Ann. Neurol. 22: 52-59, 1987). HSV-1 infections in immunmocompromised patients are characterized by severe, chronic and often extensive lesions of mucous membranes (Snoeck, Inte. J. Antimicrobiol. Agents 16: 157-159, 2000).

Currently, two classes of antiviral drugs are utilized clinically against herpes infections. The first class is represented by nucleoside analogs such as acyclovir and its prodrug derivatives (e.g. valaciclovir and famciclovir) and adenine arabinoside (ara-A). Acyclovir inhibits viral DNA synthesis selectively in HSV-infected cells. Acyclovir is activated by the viral thymidine kinase and is phosphorylated to acyclovir-triphosphate which is incorporated into the DNA chain by the viral DNA polymerase.

The second class of antiviral drugs are direct HSV DNA polymerase inhibitors, such as phosphonoformate (Foscovir) and phosphonoacetate. Resistance to these drugs arises from mutation in the thymidine kinase and in the DNA polymerase.

Xia-Ku-Cao, the fruitspikes of Prunella vulgaris L., is used in Chinese medicine for the treatment of headache with vertigo, acute conjunctivitis, lymph node tuberculosis, goiter mastitis and hypertension (Chang et al., World Scientific, Singapore, p. 964, 1987). Recently, the water extract of P. vulgaris was reported to have anti-HSV activity (Zheng, Chung His I Chieh Ho Tsa Chih 10: 39-41, 1990). Xu and coauthors reported that the polysaccharide component in P. vulgaris had potent anti-HSV activities (Xu et al., Antiviral Res. 44: 43-54, 1999). The antiviral activities of P. vulgaris have also been reported (Kageyama et al., Antiviral Chem. Chemother. 11:157-164, 2000; Tabba et al., Antiviral Res. 11: 263-274, 1989; Yamasaki et al., Biol. Pharm. Bull. 21: 829-833, 1998).

On the other hand, Tabba et al reported isolation of an anti-HIV component as an aqueous extract of this herb. This extract was characterized as a sulfated polysaccharide and was named prunellin (Tabba et al., Antiviral Res. 11: 263-274, 1989). Subsequent studies continued to identify P. vulgaris to have anti-HIV, HIVintegrase inhibitory, and HIV-protease inhibitory properties (Yamazaki et al., Biol. Pharm. Bull. 21: 829-833, 1998; Yao et al., Virology 187: 56-62, 1992; Collins, Life Sci. 60: PL345-351, 1997; Au et al., Life Sci. 68: 1687-1694, 2001; Kageyama et al., Antiviral Chem. Chemother. 11:157-164, 2000). Preliminary evaluation of the mechanism of inhibition of HIV-1 infection in vitro by purified extract of P. Vulgaris suggested that HIV-1 infection was antagonized by preventing viral attachment to the CD4 receptor (Lam et al., Life Sci. 67: 2889-2896, 2000).

Tabba et al. (Antiviral Res. 11: 263-274, 1989) and Xu et al. (Antiviral Res. 44: 43-54, 1999) report that the bioactive polysaccharide constituents which have the characteristics of dark brown color or obvious ultraviolet absorption were not thought to be “pure” polysaccharides. Moreover, in all reports, detailed chemical composition and structural chemical properties of these bioactive polysaceharide components have not been reported so far. The nature of the active ingredients which are responsible for expression of antiviral activities of P. vulgaris remains unclear.

Over the past decade, the incidence and severity of herpes infections have increased due to the increase in the number of immunocompromised patients produced by aggressive chemotherapy regimens, expanded organ transplantation, and the rising incidence of human immunodeficiency virus (HIV) infection. This change in disease pattern and the increase in frequency of drug use have resulted in the emergence of acyclovir- and other nucleoside analog-resistant herpes infections, and a need for new and useful antiviral agents, especially those with a different mode of action than acyclovir.

The inhibitory effects of polyanionic substances on the replication of herpes simplex virus (HSV) and other viruses were reported almost four decades ago. However, these observations did not generate much interest, because the antiviral action of the compounds was considered to be largely nonspecific. Shortly after the identification of human immunodeficiency virus (HIV) as the causative agent of the acquired immune deficiency syndrome (AIDS) in 1984, heparin and other anionic (sulfated) polysaccharides were found to be potent inhibitors of HIV-I replication in cell culture. Since 1988, the activity spectrum of the anionic polysaccharides has been shown to extend to various enveloped viruses, including viruses that emerge as opportunistic pathogens (e.g., herpes simplex virus (HSV) and cytomegalovirus (CMV)) in immunosuppressed (e.g., AIDS) patients. As potential anti-viral drug candidates, anionic polysaccharides offer a number of promising features.

Accordingly, there is still a need in the art for effective compounds and methods for the prevention and treatment of the cytopathogenic effects caused by each of the various forms of viral infections caused by enveloped viruses.

### SUMMARY OF THE INVENTION

In accordance with the present invention, pharmaceutical compositions which can be extracted and purified from the spikes of Prunella vulgaris are identified as effective agents for treatment of the cytopathogenic effects of an enveloped virus in mammals. Invention compositions have a molecular weight of less than 10 kDa, e.g., about 8.5 kDa and comprise a lignin-carbohydrate complex (Prunella vulgaris polysaccharide (PVP) complex) as an active ingredient having antiviral activities. In one embodiment of the invention, the ratio of lignin to carbohydrate in the complex is about 2:1. The lignin in the complex is composed of vanillin, syringaldehyde, p-hydroxybenzaldehyde and oxidation derivatives thereof. The carbohydrate in the complex is composed of glucose, arabinose, xylose, rhamnose, mannose, galactose, and galacturonic acid.

In accordance with yet another embodiment of the invention, it has been discovered that the purified compositions of the invention are effective agents for treatment of the cytopathogenic effects of an enveloped virus in mammals exposed to immunosuppressive regimens. Antiviral assay and analysis of the constituents from P. vulgaris reveal that the lignin-carbohydrate complex of the invention composition plays an important role in the direct inhibition of HSV-1 with an IC₅₀ of about 18 μg/ml. Invention composition has a direct virucidal effect and impedes the adsorption of the virus on Vero cells. It also inhibits the binding or adsorption and penetration of virus into Vero cells and therefore blocks cell to cell viral infection.

In accordance with yet another embodiment of the invention, the anti-HSV activities of the invention composition on wild type HSV-1 and the gC-deficient mutant (HSV-1 lacking the envelop glycoprotein gC) are provided. The IC₅₀ of the invention composition is much lower as compared to the IC₅₀ of heparin, against the wild type HSV-1 and gC-deficient mutant. Thus the invention composition is a potent anti-HSV-1 agent, as compared to heparin. The invention composition also prevents penetration of wild-type HSV-1 into Vero cells better than heparin. In the gC-deficient mutant, the prevention of penetration of HSV-1 into Vero cells by the invention composition is also observed. However, heparin shows no effects on the prevention of penetration of gC-deficient mutant into Vero cells. These data indicate that the invention compositions act on the penetration effects of viral gD protein as well as gC protein.

In accordance with yet another embodiment of the invention, invention compositions are found to have significant in vivo anti-HSV-1 and anti-HSV-2 activity. It is also demonstrated that 5% and higher concentration of the invention composition in cream formula has in vivo anti-HSV-1 and anti-HSV-2 therapeutic effects. All these observations suggest that invention compositions are useful as an effective anti-HSV drug.

In accordance with a further embodiment of the invention, methods for producing invention compositions comprising lignin-carbohydrate complex and uses thereof are provided. It has been discovered that invention compositions can be used for treatment of the cytopathogenic effects of an enveloped virus and related indications in mammals by employing an effective amount of the invention compositions as the active agent. It has also been discovered that the purified compositions of the invention are effective agents for protecting mammals from reactivated viral infection during or following treatment with chemotherapeutic agents.

### BRIEF DESCRIPTION OF THE FIGURES

FIG. 1 presents a representative method for extraction and isolation of an active anti-herpes extract from the spikes of Prunella vulgaris.

FIG. 2 presents a representative separation of active anti-herpes materials by a Sephadex G-50 column.

FIG. 3 collectively presents the HPLC analysis of anti-herpes preparations from Prunella vulgaris. Thus, FIG. 3A presents the HPLC elution profile of an aqueous extract; FIG. 3B presents the HPLC elution profile of a partially purified extract (PVP; see Example 1), and FIG. 3C presents the HPLC elution profile of Fraction E (see Example 3).

FIG. 4 illustrates the lack of cytotoxicity of the Prunella vulgaris aqueous extracts up to 500 μg/ml.

FIG. 5 illustrates the further separation of active anti-herpes materials by a BioGel P4 column.

FIG. 6 presents the HPLC profile of the purified Prunella vulgaris polysaccharide.

FIG. 7 presents a graph presenting an estimation of the molecular mass of the Prunella vulgaris polysaccharide by HPLC.

FIG. 8 presents a gel exclusion chromatographic pattern on Sepharose CL-6B of PVP-2, ♦, carbohydrate (490 nm); ∘, uronic acid (520 nm); ●, protein (280 nm). Vo, void volume; Vi, inner volume.

FIG. 9 illustrates the affect of protease treatment and periodate oxidation on the anti-HSV-1 activity of PVP-2b and various derivatives thereof. ●, PVP-2b; ▪, PVP-2b-PR (protease digested product of PVP-2b); PVP-2b-SD (periodate oxidized product of PVP-2b). The figure shows that there was no reduction of activity for PVP-2b-PR, in comparison with the untreated sample PVP-2b, however, PVP-2b-SD had substantially reduced activity.

FIG. 10 illustrates the effects of PVP-2b on adsorption of HSV-1 into Vero cells at 37° C. and 4° C. ●, 37° C.; ∘, 4° C. The IC₅₀ values were found to be 7.4 μg/ml at 37° C. and 6.0 μg/ml at 4° C. The virus was inhibited significantly by the sample at relatively high concentration at 4° C. and 37° C. The viral inhibition was very low for the sample at low concentration.

FIG. 11 collectively presents immunoprecipitation results related to isolation and identification of HSV-1 proteins that bind to P. vulgaris polysaccharide (PVP). ³⁵S-methionine labled HSV-1 infected (FIG. 11A) or mock-infected Vero cells (FIG. 11B) were lysed by Nonidet P40 and sodium deoxycholate and the lysate was applied to a PVP-Sepharose column. Following washing, the bound proteins were eluted with 0.5% PVP. Aliquots were immunoprecipitated by rabbit anti-gC and anti-gD antibodies. Immuno-precipitates were recovered by Protein A-agarose beads and subsequently analyzed by SDS-PAGE and autoradiography.

### DETAILED DESCRIPTION OF THE INVENTION

In accordance with the present invention, there are provided compositions in substantially purified form, said compositions comprising a lignin-carbohydrate complex (PVP complex). The ratio of lignin to carbohydrate in invention compositions is about 2:1. The lignin in the complex is composed of oxidized derivatives of vanillin, syringaldehyde, and p-hydroxybenzaldehyde. The carbohydrate in the complex is characterized as:

(1) water soluble polyanionic polysaccharide(s) comprising glucose, galactose and xylose, wherein glucose is the major constituent as analyzed by paper chromatography, wherein the molar ratio of glucose, relative to galactose, is at least 3 fold, and wherein the relative abundance of the polysaccharides is glucose>galactose˜mannose˜galacturonic acid>xylose˜rhamnose˜arabinose;

(2) having an elemental content of 30-35% carbon, preferable 31%, more prefeable 30.78%; 3-4% hydrogen, preferable 3.1%, more preferable 3.05%; 0.5-1% nitrogen, prefeable 0.7%, more preferable 0.66%; and 2-3% sulfur, preferable 2.7%, more preferable 2.69%;

(3) containing about 42% (w/w) carbohydrate, expressed as glucuronic acid; and

(4) containing about 7.5% (w/w) uronic acid, expressed as glucuronic acid.

Invention compositions comprising lignin-carbohydrate complex (PVP complex) can be further characterized as:

(1) being effective for the treatment of the cytopathogenic effects of an enveloped virus in a mammal, and being able to inhibit viral infection both before virus binding and after virus binding and penetration;

(2) being non-proteinaceous and having a molecular mass less than about 10 kDa (e.g., about 8.5 kDa);

(3) having little or no anti-coagulant activity as measured by the prothrombin time test, and having substantially little or no in vivo toxicity;

(4) being stable to exposure to temperatures in the range of about 95-100° C. for 4 hours and being substantially insoluble in methanol, ethanol, butanol, acetone, and chloroform;

(5) having a pH of 5.5 when dispersed in an aqueous solution at a concentration of about 1 mg/ml; capable of binding to Alcian blue and to DEAE Sepharose at neutral pH, and having a strong UV absorption peak at 202 nm with a shoulder at 280 nm extending to 380 nm when dispersed in aqueous medium; and

(6) having a retention time of 3.56 min when subjected to reversed-phase high pressure liquid chromatography (HPLC) on a C18 column (25 cm×4.6 mm ID, 5μ, Supelcosil LC-18, Sigma) and eluted with 5% water: 95% acetonitrile at a flow rate of 0.3 ml/min.

The novel compositions of the present invention can be prepared by a variety of established methods for both extraction and purification. One such method is the one described in Examples 1-3 in the present specification. In another approach, for example, invention compositions can be obtained from cells of the plant Prunella vulgaris, by purifying the invention composition by contacting an extract from Prunella vulgaris with an anion exchange material which selectively binds negatively charged materials, and recovering the invention composition from the anion exchange material.

The present invention is also directed to pharmaceutical formulations suitable for the treatment of the cytopathogenic effects of an enveloped virus in a mammal in need thereof, which contains an effective amount of invention composition, with or without an appropriate pharmaceutically acceptable carrier therefor. Examples of enveloped virus include Herpes simplex virus type 1 and 2, human immunodeficiency virus type 1 (HIV-1), human cytomegalovirus, measles virus, mumps virus, influenza and parainfluenza virus, respiratory syncytial virus, and the like.

The present invention is also directed to pharmaceutical formulations suitable for the treatment of cytopathogenic effects of an enveloped virus, more preferably herpes simplex virus, in a mammal in need thereof, which formulation contains an effective amount of said purified composition or an effective amount of said purified composition together with a pharmaceutically acceptable carrier therefor.

The present invention is also directed to methods for treating the cytopathogenic effects of an enveloped virus which comprises administering to a mammal in need thereof an effective amount of the above-described pharmaceutical formulation, or of said purified composition, optionally with a pharmaceutically acceptable carrier. More particularly, the mammal to be treated is a human who has been diagnosed as having an infection caused by said enveloped virus. Most particularly the mammal to be treated is a human who has been specifically diagnosed as having herpes simplex virus.

In accordance with another embodiment of the present invention, there are provided methods for the treatment of the cytopathogenic effects of an enveloped virus in a mammal. Invention methods comprise administering to a mammal in need thereof an effective amount of the above-described purified composition.

As used herein, “PVP” refers to prunella vulgaris polysaccharide. “PVP complex” refers to lignin-carbohydrate complex according to the invention. The term “PVP complex” is interchangeable with the terms “lignin-carbohydrate complex”, “PVP”, “PVP-2b” and “PVP compound”, meaning prunella vulgaris polysaccharide complex comprising lignin and carbohydrate.

As used herein, “mammal” refers to humans as well as other mammals, and includes animals of economic importance such as bovine, ovine, and porcine animals. The preferred mammal contemplated for treatment according to the invention is a human. Adults as well as non-adults (i.e., neo-nates, pre-pubescent mammals, and the like) are contemplated for treatment in accordance with the invention.

As used herein, the phrase “cytopathogenic effect of an enveloped virus” refers to the abnormal condition of a cell caused by the infection by an enveloped virus. As noted above, viral infections caused by enveloped viruses are a heterogenous group of disorders characterized by viral infection in host cells. Viral infections caused by enveloped viruses can be acquired as a direct result of exposure to an enveloped virus, or reactivation of an enveloped virus infection in a seropositive mammal, and which reactivation may be induced by exposure to a broad category of agents, including but not limited to a variety of environmental factors (e.g., stress), immunocompromising regimens (i.e., pertaining to an immune response that has been weakened by a disease or a chemotherapy agent or an immunosuppressive agent), weakened immunocompetence arising from other viral infections (such as HIV and the like), age onset, and the like. As described herein, disorders characterized by viral infections in cells include infections of cultured cells (i.e., lytic infections, persistent infections, latent infections, transforming infections, abortive infections, and the like), infectious disorders, conditions and diseases (i.e., acute infections, inapparent or silent infections, chronic and persistent infections, latent infections, slowly progressive diseases, virus-induced tumours, and the like), and the like.

The compositions of the present invention are preferably present in a purified form when administered to a patient. When invention compositions are obtained by extraction from plant spikes, it is desirable to separate soluble extract from (residual) particulate matter by appropriate means (e.g., filtration, centrifugation, or other suitable separation techniques). The utility of invention compositions as a therapeutic agent is enhanced by greater purification. Greater dosages may be necessary when less pure forms of the extract are employed.

Invention compositions are preferably substantially free from heavy metals, contaminating plant materials, contaminating microorganisms, oxalic acid or precursors of oxalic acid or any other contaminants which may be present in a preparation which can be derived from plant material.

Invention compositions can also be used to inhibit the cytopathogenic effects of enveloped viruses (e.g., HSV) in mammals in need thereof, more particularly in humans.

Invention compositions are produced by (a) extracting spikes of Prunella vulgaris; (b) precipitating and purifying the extract obtained from (a); (c) fractionating purified extract of (b); and (d) analyzing purified fraction of (c) for antiviral activities.

Although isolation of invention compositions from the spikes of Prunella vulgaris plants is the presently most practical method for obtaining such materials, the present invention also contemplates obtaining such materials from other sources such as other plants which may contain recoverable amounts of compositions having the properties described herein. Other plants contemplated include species within the subfamily Nepetoideae, of which Prunella is a member. It is also possible that invention compositions could be obtained by culturing plant cells, such as Prunella vulgaris cells, in vitro and extracting the active ingredients from the cells or recovering the active ingredients from the cell culture medium.

As used herein, the term “extract” means the active ingredients isolated from spikes or other parts of Prunella vulgaris or other natural sources including but not limited to all varieties, species, hybrids or genera of the plant regardless of the exact structure of the active ingredients, form or method of preparation or method of isolation. The term “extract” is also intended to encompass salts, complexes and/or derivatives of the extract which possess the above-described biological characteristics or therapeutic indication. The term “extract” is also intended to cover synthetically or biologically produced analogs, homologs and mimics with the same or similar characteristics yielding the same or similar biological effects of the present invention.

The purified compositions contemplated for use herein include purified extract fractions having the properties described herein from any plant or species, preferably Prunella vulgaris, in natural or in variant form, and from any source, whether natural, synthetic, or recombinant. Also included within the scope of the present invention are analogs, homologs and mimics of the above-described purified compositions.

The present invention also contemplates the use of synthetic preparations having the characteristics of invention compositions. Such synthetic preparations could be prepared based on the chemical structure and/or functional properties of the above-described compositions of the present invention. Also contemplated are analogs, homologs and mimics of the chemical structure of the invention compositions and having the functional properties of compositions according to the present invention.

As used herein, reference to “analogs, homologs and mimics” of the invention compositions embraces compounds which differ from the structure of invention compositions by as little as the addition and/or replacement and/or deletion of one or more residues thereof, to compounds which have no apparent structural similarity. Such compounds in all instances, however, have substantially the same activity as invention compositions. Thus, “analogs” refers to compounds having the same basic structure as invention compositions, but differing in several residues; “homologs” refers to compounds which differ from invention compositions by the addition and/or deletion and/or replacement of a limited number of residues; and “mimics” refers to compounds which have no specific structural similarity with respect to invention compositions. Indeed, a mimic need not even be a polyanionic carbohydrate, but such compound will display the biological activity characteristics of the invention composition.

As used herein, “treatment” refers to therapeutic and prophylactic treatment. Those in need of treatment include those already with enveloped virus infections as well as those in which treatment of the enveloped virus infection has failed.

Invention compositions described for use herein can be delivered in a suitable vehicle, thereby rendering such composition amenable to oral delivery, transdermal delivery, subcutaneous delivery (e.g., intravenous delivery, intramuscular delivery, intraarterial delivery, intraperitoneal delivery, and the like), topical delivery, inhalation delivery, osmotic pump, and the like. Depending on the mode of delivery employed, the above-described composition can be delivered in a variety of pharmaceutically acceptable forms. For example, the above-described composition can be delivered in the form of a solid, solution, emulsion, dispersion, micelle, liposome, and the like.

Pharmaceutical formulations contemplated for use in the practice of the present invention contain invention composition in admixture with an organic or inorganic carrier or excipient suitable for enteral or parenteral applications. The active ingredient may be Compounded, for example, with the usual non-toxix, pharmaceutically acceptable carries for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. The active compounds contemplated for use herein are included in the pharmaceutical formulation in an amount sufficient to produce the desired effect upon the target enveloped viral disease.

Pharmaceutical formulations containing the active compound contemplated herein may be in a form suitable for oral use, for example, as tablets, troches, lozenges, aqueous or oily suspensions, dispersible powders or granules, emulsions, hard or soft capsules, or syrups or elixirs. Formulations intended for oral use may be prepared according to any method known in the art for the manufacture of pharmaceutical formulation. In addition, such formulations may contain one or more agents selected from a sweetening agent (such as sucrose, lactose, or saccharin), flavoring agents (such as peppermint, oil of wintergreen or cherry), colouring agents and preserving agents, and the like, in order to provide pharmaceutically elegant and palatable preparations. Tablets containing the active components in admixture with non-toxic pharmaceutically acceptable excipients may also be manufactured by known methods. The excipients used may be, for example, (1) inert diluents such as calcium carbonate, lactose, calcium phosphate, sodium phosphate, and the like; (2) granulating and disintegrating agents such as corn starch, potato starch, alginic acid, and the like; (3) binding agents such as gum tragacanth, corn starch, gelatin, acacia, and the like; and (4) lubricating agents such as magnesium stearate, stearic acid, talc, and the like. The tablets may be uncoated or they may be coated by known techniques to delay disintegration and absorption in the gastrointestinal tract, thereby providing sustained action over a longer period. For example, a time delay material such as glyceryl monostearate or glyceryl distrearate may be employed. They may also be coated by the techniques described in the U.S. Pat. Nos. 4,256,108; 4,160,452; and 4,265,874, to form osmotic therapeutic tablets for controlled release.

In some cases, formulations for oral use may be in the form of hard gelatin capsules wherein the active components are mixed with an inert solid diluent, for example, calcium carbonate, calcium phosphate, kaolin, or the like. They may also be in the form of soft gelatin capsules wherein the active components are mixed with water or an oil medium, for example; peanut oil, liquid paraffin, or olive oil.

The pharmaceutical formulation may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

It may be desirable to administer in conjunction with the invention composition other viral DNA synthesis inhibitors or DNA polymerase inhibitors that promote synergistic therapeutic and prophylactic effects.

The treatment regimen or pattern of administration of the agents may be one with simultaneous administration of an agent which counteracts the effects of invention compositions, and invention compositions. In addition, the treatment regimen may be phasic with an alternating pattern of administration of one agent followed at a later time by the administration of the second agent. Phasic administration includes multiple administrations of one agent followed by multiple administrations of the second agent. The sequence that the agents are administered in and the lengths of each period of administration would be as deemed appropriate by the practitioner.

Invention compositions can also be suitably administered employing sustained-release systems. Suitable examples of sustained-release compositions include semi-permeable polymer matrices in the form of shaped articles, e.g. films, or microcapsules. Sustained-release matrices include polylactides (U.S. Pat. No. 3,773,919), copolymers of L-glutamic acid and gamma-ethyl-L-glutamate (Sidman et al., Biopolymers, 22: 547-556 (1983)), poly(2-hydroxyethyl-methacrylate) (Langer et al., J. Biomed. Mater. Res., 15: 267-277 (1981)), ethylene vinyl acetate (Langer et al., supra) or poly-D-(−)-3 hydroxybutyric acid (EP133,988), and the like. Sustained-release formulations containing invention compositions also include liposomally entrapped compositions according to the invention. Liposomes are prepared by methods known in the art (see, for example, DE 3,218,121; U.S. Pat. Nos. 4,485,045 and 4,545,545).

For parenteral administration, in one embodiment, invention compositions are formulated by mixing in a unit dosage injectable form (solution, suspension, or emulsion), with a pharmaceutically acceptable carrier, i.e., one that is non-toxic to recipients at the dosages and concentrations employed and is compatible with other ingredients of the formulation. The formulation preferably does not include oxidizing agents and other compounds that are known to be deleterious to polypeptides.

Generally, the formulations are prepared by contacting invention compositions uniformly and intimately with liquid carriers or finely divided solid carriers or both. Then, if necessary, the product is shaped into the desired form. Preferably the carrier is a parenteral carrier, more preferably a solution that is isotonic with the blood of the recipient. Examples include water, saline, Ringers solution, dextrose solution, and the like. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as poly-vinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxmers, or PEG. Invention compositions are typically formulated in such vehicles according to clinically relevant/acceptable protocol. It will be understood that use of certain of the foregoing excipients, carriers, or stabilizers will result in the formation of salts of invention compositions.

In addition, invention compositions are suitably formulated in an acceptable carrier vehicle to form a pharmaceutical formulation, preferably one that does not contain cells. In one embodiment, the buffer used for formulation will depend on whether the resulting formulation will be employed immediately upon mixing or stored for later use. If employed immediately, invention compositions can be formulated in mannitol, glycine, and phosphate at an appropriate pH. If this mixture is to be stored, it is preferably formulated in a buffer at an appropriate pH, in the optional further presence of a surfactant that increases the solubility of invention compositions at this pH. The final preparation may be a stable liquid or a lyophilized solid.

While invention compositions can be formulated in any way suitable for administration, presently preferred formulations contain about 2-20 mg/mL of invention composition, about 2-50 mg/ml of an osmolyte, about 1-15 mg/ml of a stabilizer, and a buffered solution at about pH 5-6, more preferably pH about 5-5.5. Preferably, the osmolyte is an inorganic salt at a concentration of about 2-10 mg/ml or a sugar alcohol at a concentration of about 40-50 mg/ml, the stabilizer is benzyl alcohol or phenol, or both, and the buffered solution is an acetate-buffered solution. Even more prefered are formulations wherein the osmolyte is sodium chloride and the acetic acid salt is sodium acetate. Still more preferably, the amount of invention composition is about 8-12 mg/ml, the amount of sodium chloride is about 5-6 mg/ml, the amount of benzyl alcohol is about 8-10 mg/ml, the amount of phenol is about 2-3 mg/ml, and the amount of sodium acetate is about 50 mM so that the pH is about 5.4. Additionally, the formulation can contain about 1-5 mg/ml of a surfactant, preferably polysorbate or poloxamer, in an amount of about 1-3 mg/ml. Alternatively, for the preparation of invention formulation, invention composition is suitably dissolved at 5 mg/ml in 10 mM citrate buffer and 126 mM NaCl at pH 6.

Invention compositions to be used for therapeutic use must be sterile. Sterility is readily accomplished by filtration through sterile filtration membranes (e.g., 0.2 micron membranes). Therapeutic compositions and formulations according to the invention generally are placed into a container having a sterile access port, for example, a vial having a stopper pierceable by a hypodermic injection needle.

Invention compositions and formulations ordinarily will be stored in unite or multi-dose containers, for example, sealed ampules or vials, as an aqueous solution, or as a lyophilized formulation for reconstitution. As an example of a lyophilized formulation, 10 ml vials are filled with 5 ml of sterile-filtered aqueous solution of composition according to the invention, and the resulting mixture is lyophilized. The infusion solution is prepared by reconstituting the lyophilized material in bacteriostatic Water-for-Injection.

Typical daily doses of the active component, in general, lie within the range of from about 10 μg up to about 1g per kg body weight, and, preferably within the range of from about 100 μg up to about 500 mg per kg body weight and can be administered up to five times daily, for example. Presently preferred daily doses lie within the range of from about 1 mg up to about 50 mg per kg body weight. Typically, the active compound will be present in an amount of 0.1 to 90 percent by weight of the pharmaceutical formulation, preferably 0.5 to 50 percent by weight of the pharmaceutical formulation, for example. Where one will operate within the above ranges will vary depending on the route of administration and on a variety of considerations, such as, for example, the age of the patient, the size of the patient, other dysfunctions of the patient, what, if any, other medications the patient may be taking, and the like.

As a general proposition, the total pharmaceutically effective amount of invention composition administered parenterally per dose will be an amount sufficient to provide a therapeutic effect without inducing a significant level of toxicity. Since individual subjects may present a wide variation in severity of symptoms and each active ingredient has its unique therapeutic characteristics, it is up to the practitioner to determine a subject's response to treatment and vary the dosages accordingly.

Based on the in vitro data presented herein, a concentration of 10 μg to 50 mg of invention composition (see Example 5) per gram of cream or ointment is expected to be effective for topical application in preventing the cytopathogenicity of the herpes simplex virus.

In accordance with another embodiment of the present invention, there are provided methods for the treatment of the cytopathogenic effects of an enveloped virus and related indications in mammals in need thereof, said method comprising administering an effective amount of invention composition to said mammal.

Patients who present the cytopathogenic effects of an enveloped virus contemplated for treatment in accordance with the present invention are those testing seropositive to an enveloped virus, preferably, those who are diagnosed with HSV, VZV or HIV infections.

In accordance with yet another embodiment of the present invention, there are provided methods for the treatment of the cytopathogenic effects of an enveloped virus in mammals exposed to immunosuppressive regimens, said method comprising administering an effective amount of invention composition to said mammal.

Patients who present the cytopathogenic effects of an enveloped virus and exposed to immunosuppressive regimens contemplated for treatment in accordance with the present invention are those whose normal immunotolerance is compromised by exposure to immunosuppressive agents following, for example, organ transplantation, xenotransplantation, subjects who have undergone chemotherapy (e.g., cancer patients), and the like.

In accordance with still another embodiment of the present invention, there are provided methods for protecting mammals exposed to or following treatment with chemotherapeutic agents, from the cytopathogenic effects of a reactivated enveloped virus infection, said method comprising administering an effective amount of invention composition to said mammal.

Patients for whom protection from the reactivated cytopathogenic effects of an enveloped virus during or following treatment to chemotherapeutic agents is indicated include patients suffering from any disease which is commonly treated by the administration of chemotherapeutic agents, e.g., post organ transplant, nephrotic syndrome, cancer patients, and the like.

In accordance with a further embodiment of the present invention, there are provided methods for preparing novel extract fractions of Prunella vulgaris having antiviral action and formulations containing said extract fractions. See, for example, the methods described in the following Examples provided herein.

The extract or extract fractions that are active in preventing the cytopathogenic effects of an enveloped virus could be used to treat the cytopathogenic effects of enveloped viral infections in mammals, most preferably humans, in the following way:

(i) Since the untreated aqueous extract has been administered to mammalian cells up to concentrations as high as 0.5 mg/ml (see Example 14) with no apparent side effects, it could also be ingested by human subjects and may reduce the potential for viral associated disease.

(ii) The compound that is active against the enveloped virus could also be administered intravenously to patients with viral infection after the compound has been further purified. The compound may be more effective when administered I.V. than orally since it is unlikely that 100% of the active compound would be absorbed from the gastrointestinal tract.

(iii) It is also conceivable that the purified compound active against the enveloped virus could be administered into the spinal fluid and may prevent the potential for enveloped viral associated disease in patients with an enveloped viral infection.

It is contemplated that the extract will be formulated into a pharmaceutical composition comprising an effective amount of the extract with or without a pharmaceutically acceptable carrier (as previously described). All references and patents cited herein are hereby incorporated by reference.

A number of natural products are known to have inhibitory effects on herpes simplex virus. Musci and Pragai (see Experientia 41: 6 (1985)) showed the inhibitory effect of four flavonoids, i.e., quercetin, quercitrin, rutin, and hesperidine, on HSV-1 and Suid (alpha) HSV-1 (pseudorabies virus). A direct relationship between viral inhibition and the ability of flavonoids to increase cyclic AMP in the host cells was observed, suggesting flavonoids exert their antiviral effects via cyclic nucleotide metabolism. Wleklik et al. (see Acta Virol. 32: 522 (1988)) further showed that hydroxylation at positions 3, 5, 7, 3′, and 4′ of flavonoids was associated with the highest anti-herpes activity. Hayashi et al. (see Antimicrob. Agents Chemother. 36: 1890-1893 (1992)) described inhibition of HSV-1 and HSV-2 replication in Vero cells by a bifavanone ginkgetin isolated from Cepalotaxus drupacea. The IC₅₀ (50% inhibition concentration) against HSV-1 was 0.91 μg/ml. Ginkgetin suppressed viral protein synthesis and had no effect on the binding and penetration of HSV-1 into cells. Barnard et al. (see Chemother. 39:203-211 (1993)) described a flavonoid polymer of 2100 Da that inhibited HSV penetration into cells.

Polysaccharides are known to affect the growth of animal viruses (see Shannan, W. M. in G. J. Galasso, T. C. Merigan, and R. A. Buchanon (ed.), Antiviral agents and viral diseases of man. Raven Press, New York (1984) at p. 55-121). In particular, anionic polysaccharides, such as heparin, dextran sulfate, carrageenans, pentosan polysulfate, fucoidan, and sulfated xylogalactans, are potent inhibitors of herpes virus binding to host cells (see, for example, Gonalez et al. in Antimicrob. Agents Chemother. 31: 1388-1393 (1987); Baba et al. in Antimicrob. Agents Chemother. 32: 1742-1745 (1988); and Damonte et al. Chemother. 42: 57-60 (1996)). The activity spectrum of the sulfated polysaccharides has been shown to extend to various enveloped viruses (e.g., human immunodeficiency virus type 1 (HIV-1), measles virus, mumps virus, influenza and parainfluenza virus, respiratory syncytial virus (HSV) and cytomegalovirus (CMV)), including viruses that emerge as opportunistic pathogens. These polysaccharides are competitor of receptors (heparan sulfate) to viral glycoproteins. Herold et al. (see J. Virol. 70: 3451-3469 (1996) showed that N-sulfations and the presence of carboxy groups on heparin are key determinants for HSV-1 and HSV-2 interactions with host cells. However, these polysaccharides also have anti-coagulant activity and are therefore unsuitable as anti-herpes drugs.

Some plant proteins are known to have anti-herpes activities. The better known ones are ribosome-inactivating proteins (e.g. pokeweed anti-viral protein (see, for example, Teltow et al. in Antimicrob. Agents Chermother. 23: 390 (1983)) and lectins (e.g. concanavalin A; see, for example, Okada, Y., and J. Kim. Virology 5: 507 (1972)). Pokeweed anti-viral protein is a 30 kDa single polypeptide that binds irreversibly to HSV and enters host cells only after binding to the virus. Following entry into the host cells, the protein inhibits protein synthesis. Concanavalin A interacts with the viral envelope to inhibit infectivity or to block exit of virus from infected cells. More recently, the anti-HSV activity of two other plant proteins and one human serum protein were described. MAP30 and GAP31 are a 30 kDa and 31 kDa protein isolated from the Chinese bitter melon Momordica charantia and a Himalayan tree Gelonium multiflorum, respectively (see, for example, Bourinbaiar, A. S., and S. Lee-Huang, in Biochem. Biophys. Res. Comm. 219: 923-929 (1996)). Both proteins showed IC₅₀ against HSV-1 and HSV-2 in the 0.1 to 0.5 μM range. The mode of action of MAP30 and GAP31 is not known. The high density serum apolipoprotein A-I was found to inhibit HSV-induced cell fusion at 1 μM (see Srinivas, R. V., et al., Virology 176:48-57 (1990)). An 18 amino acid synthetic peptide analog to apolipoprotein A-1 inhibited penetration of virus into cells but did not prevent virus adsorption.

Other anti-herpes natural products include terpenoids and tannins. Terpenoids (e.g. glycyrrhizic acid, see, for example, Vanden Berghe, D. A., et al., in Bull. Inst. Pasteur 84: 101 (1986)) inhibit HSV replication (see Hudson, J. B. in Antiviral compounds from plants. CRC Press, Inc. Boca Raton, Fla. (1990)). Tannins are thought to inhibite viral adsorption (see, for example, Fukudri, K., et al. in Antiviral Res. 11: 285-297 (1989)). Xu et al. (see Heterocycles 38: 167-175 (1994)) showed that a hydrolyzable tannin, geponin, and gallic aldehyde had IC₅₀ against HSV-1 of 25 and 12.5 μg/ml, respectively.

Prunella vulgaris, a perennial plant commonly found in China, the British Isles, and Europe, has been used as an astringent for internal and external purposes (see, for example, Grieve, M. in A modern herbal. Dover Publications, NY. (1973)), as a crude anticancer drug (see, for example, Lee, H., and J. Y. Lin. in Mutation Res. 204: 229-234 (1988)), and as a herbal remedy to lower high blood pressure (see, for example, Namba, T. in The encyclopedia of Wakan-Yaku (traditional Sino-Japanese medicines), Vol II, p. 120-121; Hoikusha Publishing Co. Ltd. Osaka, Japan (1994)). In western herbal remedies, the plant (which is better known as “self heal”) is used in the form of hot water infusion sweetened with honey to treat sores in the mouth and throat (see, for example, Grieve, M. in A modern herbal. Dover Publications, NY. (1973)). Zheng (see ChungHsi-I-Chieh-Ho-Tsa-Chih. 10: 39-41 (1990)) reported the use of a crude aqueous extract of Prunella vulgaris in clinical treatment of herpetic keratitis with some success. Of the 78 patients who received eye drops containing crude extracts of Prunella vulgaris and Pyrrosia lingua, 38 were reported to be cured, 37 showed improvement, and 3 did not respond. A crude aqueous extract of Prunella vulgaris contained no detectable anti-coagulant activity (see Zeng, F.-Q. M. Sc. Thesis. National University of Singapore (1996)). Hence, while there is some evidence that Prunella vulgaris is an anti-herpes plant, the active anti-herpes components are not known.

HSV-1, HSV-2, and VZV belong to the human alpha-herpes viruses, while cytomegalovirus and Epstein-Barr virus belong to the beta-herpes and gamma-herpes virus, respectively. HSV are large (180-200 nm in diameter) enveloped viruses containing double stranded DNA. The DNA core is surrounded by an icosahedral capsid containing 162 capsomers. The capsid is in turn, enclosed by a glycoprotein-containing envelope, composed of at least 11 known glycoproteins (gB, gC, gD, gE, gG, gH, gI, gJ, gK, gL, and gM), a number of which are responsible for viral attachment to cells, and the fusion of infected cells. Between the envelope and the capsid is the tegument, which contains other viral proteins.

HSV infection of host cells is a multi-step event beginning with binding of the virion to cell surface proteoglycans. Heparin sulfate moieties on proteoglycans are the site of virus binding, although HSV can also bind to chondroitin sulfate. Two viral glycoproteins, gC and gB, are responsible for HSV attachment to heparin sulfate. gC is the principal player in the binding and when it is absent from the virion, gB mediates binding at a reduced efficiency. A subsequent step in HSV entry into the cell involves the interaction of gD with a second cell surface molecule, possibly the mannose-6-phosphate receptor (see Brunetti, C. R., et al., J. Biol. Chem 269: 17067-17074 (1994)). HSV penetration occurs by fusion of the viral envelope with the cell membrane. This fusion is pH-independent and requires the participation of at least four viral glycoproteins, gB, gD, gH, and gL. Following the fusion, viral nucleocapsid is released into the cytoplasm and is uncoated to allow the viral DNA to enter the nucleus. Viral multiplication occurs in the nucleus in an orderly fashion with the initial appearance of immediate-early proteins necessary for regulation of gene transcription, early proteins (e.g. DNA polymerase), and late proteins (structural proteins). The progeny virions are widely believed to be assembled in the nucleus and exit from the cell through the endoplasmic reticulum.

In accordance with the present invention, extracts from more than 20 plants have been screened for anti-HSV-1 activity using the standard plaque reduction assay (see Edgar, L., et al. in Manual of clinical microbiology-5th ed. A. Balows (chief ed.) American Society for Microbiology, Washington D.C. (1991), p. 1184-1191). The hot-water extract prepared from the spike of Prunella vulgaris showed good activity and was not cytotoxic. A partially purified extract (PVP) was prepared from the freeze-dried aqueous extract by ethanol precipitation. The anti-herpes extract was further purified by gel permeation column chromatography (Sephadex G-50). One fraction (Fraction E) with anti-herpes activity was collected. HPLC analysis using a reversed-phase (ODS-2) column showed that fraction E contained one major peak and two very minor peaks. Plaque reduction assay showed that PVP and Fraction E had an IC₅₀ against HSV-1 of about 18 and 10 μg/ml, respectively.

The purified extract was also active on clinical isolates and acyclovir-resistant (thymidine kinase-deficient and DNA polymerase-deficient) strains of HSV-1 and HSV-2. Preincubation of HSV-1 with the purified extract abolished the infectivity of the virus; however, pretreatment of Vero cells did not prevent HSV-1 infection, confirming that the extract prevented the early event (binding and/or penetration) of infection. In a one-step growth study, addition of PVP at 0, 2, 4, and 7.25 h after infection showed a 99, 96, 94 and 90% reduction in total (extracellular and intracellular) viral yield, respectively. These results confirm that the compound also interfered with viral replication.

Fraction E contained about 42% (w/w) carbohydrate (expressed as glucuronic acid) as determined by the phenol sulfuric acid assay (see Dubois, M., et al. 1956. Anal. Biochem. 28:350-356 (1956)). It contained about only 7.5% (w/w) uronic acid (expressed as glucuronic acid) as determined by the uronic acid assay described by Blumenkrantz and Asboe-Hansen (see Blumenkrantz, N., and G. Asboe-Hansen in Anal. Biocehm. 54: 484-489 (1973). The compound is polyanionic, as evidenced from its binding to Alcian blue (Whiteman, P. in Biochem. J. 131: 343-350 (1973)) and to DEAE Sepharose at neutral pH. Hexosamines and proteins were not detected. As analyzed by paper chromatography, the purified anti-herpes polysaccharide was identified to be composed of glucose, galactose and xylose.

The purified extract was water soluble; but was insoluble in methanol, ethanol, butanol, acetone, or chloroform. The aqueous solution (1 mg/ml) of Fraction E had a pH of 5.5. Spectrophotometry showed a strong absorption peak at 202 nm with a shoulder at 280 nm which extended to 380 nm. The molecular mass of the purified compound, estimated by HPLC with a gel filtration column, was 3,500 kDa. The polyanionic polysaccharide, prunellin, previously isolated by Tabba et al. (see Antiviral Res. 11: 263-274(1989)) had a pH of 7.4 in aqueous solution, showed an adsorption peak at 370 nm which extended to 500 nm, and has a molecular mass of 10,000 kDa. This confirms that the anti-herpes compound of Fraction E contains polyanionic carbohydrate, and is chemically different from prunellin.

In accordance with the present invention, it has been discovered that extracts of Prunella vulgaris are effective in the treatment of anti-herpes viruses. The plant is known to contain oleanolic acid, triterpene acids (ursolic acid), triterpenoids, flavonoids (rutin), fenchone, tannins, and prunellin (see, for example, Namba, T. in The encyclopedia of Wakan-Yaku (traditional Sino-Japanese medicines), Vol II, p. 120-121; Hoikusha Publishing Co. Ltd. Osaka, Japan (1994)). Prunellin is a 10 kDa anionic polysaccharide and it has been shown to inhibit the replication of human immunodeficiency virus-1 (see, for example, Tabba, H. D., in Antiviral Res. 11: 263-274 (1989), and Yao, X.-J., et al. in Virology 187: 56-62 (1992)). Rutin has been shown to have some activity against HSV (see, for example, Mucsi, I., and B. M. Pragai. in Experientia 41: 6 (1985)), however, none of the other compounds have been demonstrated to have activity against herpes viruses.

The laboratory rabbit eye and mouse ear infection models are well characterized as experimental models in HSV infections. These and other animal infection models can be used to study the utility of the present extract fraction from Prunella vulgaris in preclinical evaluations.

The Examples that follow describe extraction of Prunella vulgaris plant spikes, characteristics of the extract and the effect of the extract on inhibition of the cytopathogenic effects of herpes simplex virus.

The results described in the Examples clearly show that the P. vulgaris polysaccharide complex (PVP complex) is a more superior anti-viral agent than known anti-herpes agents. In contrast with heparin, a known anti-coagulant, PVP complex had an average prothrombin time similar to that of water, indicating that PVP complex has no or substantially no anti-coagulant activity. In addition, PVP complex was found to inhibit HSV infection before virus binding as well as after virus binding and penetration. The one-sdtep growth study (Example 11) clearly shows that even after 7.25 h post virus infection, the PVP complex could reduce virus yield by 90%. This contrasts with sodium heparin which has anti-herpes activity only when it is present at the start of the virus infection, i.e., only when the virus has not yet bound to the cells to initiate the infection cycle.

The following Examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

### EXAMPLES

### Plant Materials & General Methods

The fruitspikes of P. vulgaris were purchased from Chinese Medicinal Material Co. (China). A voucher specimen was authenticated and deposited in the herbarium (No. 2399) at the Institute of Chinese Medicine of the Chinese University of Hong Kong. Total carbohydrate, uronic acid and protein contents were determined by phenol-H₂SO₄ (Dubois et al., 1956), m-hydroxybiphenyl (Blumenkrantz et al., 1973) and Bradford's methods (Bradford, 1976) with Coomasie Brilliant Blue dye (Bio-Rad), respectively, by using galactose (Gal), galacturonic acid (GalA) and bovine serum albumin (BSA) as the respective standards. Homogeneity of active substances was analyzed by HPLC on combined columns (0.8×30 cm each) of Shodex sugar KS-850 and KS-840 (Showa Denko Co, USA) in 0.2 M NaCl, and the fractions were detected by an HP 1047A refractive index (RI) which was linked in series connection with an HP 1040 A ultraviolet diode array (UV) detector. The molecular weights of samples were estimated by calibration curve of elution volumes of standard pullulans (P-400, 200, 100, 50, 20, 10, and 5). Lignin was analyzed by alkaline nitrobenzene oxidation (Chen, 1988), and the resulting benzaldehyde derivatives (vanillin, syringaldehyde and p-hydroxybenzaldehyde) were identified by GLC-MS as the method described by Kiyohara et al. (1999) on an HP-5 capillary column (0.32 mm i.d.×30 m, 0.25 m film thickness, Hewlett-Packard). Helium (2 ml/min) was used as a carrier gas, and injector and detector temperatures were 200 and 270° C., respectively. Temperature program was: 60° C. (1 min), 60->140° C. (15° C./min), 140->250° C. (5° C./min). Contents of lignin in samples were colorimetrically determined by an improved acetyl bromide method (Dence, Springer-Verlag, Berlin: 33-61, 1992), Polysaccharides were hydrolyzed with 2 M TFA at 121° C. for 1.5 h. Component sugars of the samples were analyzed as trimethylsilyl methylglycoside derivatives by GLC (York et al., Methods Enzymol. 118: 3-40, 1986) on a HP-1 capillary column (0.25 mm i.d.×30 m, 0.2 μm film thickness, Supleco, USA); the temperature program was: 140° C. for 1 min, 140->180° C. (2° C./min), 180->275° C. (1° C./min), and 275° C. for 5.8 mm.

### Example 1

### Extraction and Fractionation of Invention Composition from Prunella vulgaris: Method 1

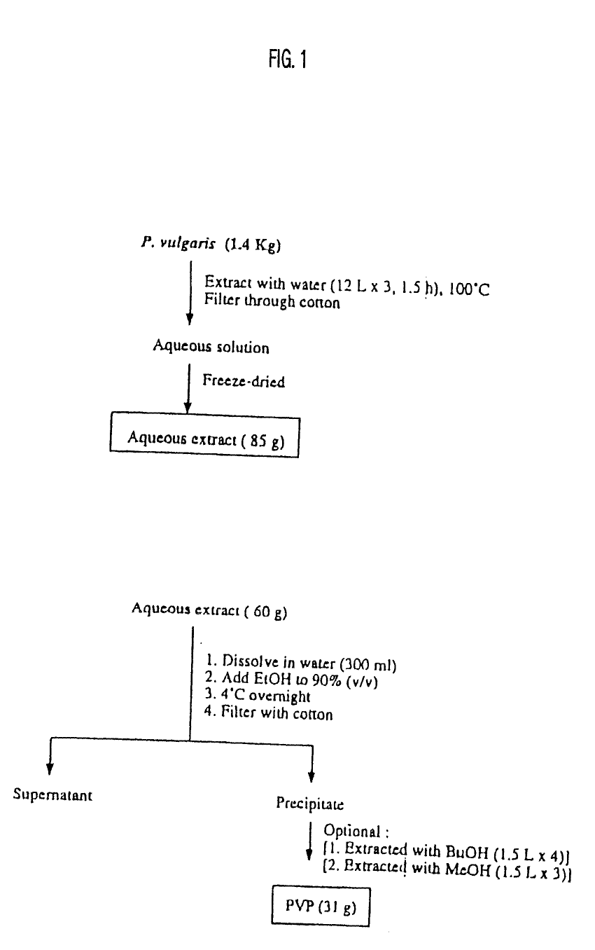

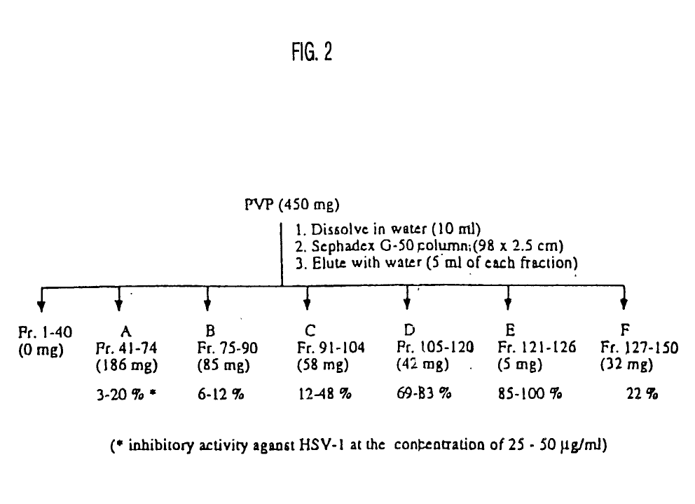

Dried spikes of Prunella vulgaris (1.4 kg) were ground into small pieces with a Waring blender. Distilled water (12 L) was added and the suspension was simmered at 95-100° C. for 90 min. The extract was decanted to a clean container and the plant was extracted two more times with water under the same conditions. The extracts were poured through a cotton cloth to remove insoluble plant materials. The volume of the clarified extracts was reduced to about 1 liter by a rotary evaporator. The condensed extract was freeze-dried. A total of 85 g of dark brown dried powder was obtained (see FIG. 1).

The anti-herpes component in the aqueous extract was precipitated by ethanol. Briefly, 30 g of the freeze-dried aqueous extract was dissolved in 300 ml of water, and ethanol was added to a final concentration of 90% (vol/vol). The mixture was incubated at 4° C. for 18 h. The precipitate was recovered by filtration through cotton and washed with 4×1.5 L of butanol, followed by 3×1.5 L of methanol. This yielded 31 g of dark brown powder, designated PVP complex (see FIG. 1). The supernatant from ethanol precipitation contained no detectable anti-herpes activity by the plaque reduction assay (see Example 3) and was discarded.

### Example 2

### Extraction and Fractionation of Invention Composition: Method 2

The extraction process was based on a method described previously by Zhang et al. (1997). Briefly, the fruitspikes of P. vulgaris (500 g) were decocted for 1 h with 10 L of H₂O and the residue was decocted again for 1 h with 8 L of H₂O. The hot water extract (PVP-0, yield: 10.6%) was obtained from lyophilization of the combined aqueous extract solution. PVP-0 was re-dissolved in 1.5 L of H₂O and then the polysaccharide components in PVP-0 were precipitated by the addition of three volumes of EtOH after water-insoluble materials had been removed by centrifugation. The resulting precipitates were dissolved in water and dialyzed against H₂O for 4 days using dialysis membrane (36 mm, Wako Chemicals Co., USA). After the non-dialyzable portion was centrifuged to isolate the insoluble materials, the supernatants were lyophilized to obtain a water-soluble crude polysaccharide fraction (PVP-1, yield: 2.8%).

PVP-1 was fractionated by cetyltrimethylaminium bromide (CTAB) method (Yamada et al., 1984) to afford the acidic polysaccharide (PVP-2), weakly acidic polysaccharide (PVP-3) and the neutral polysaccharide (PVP-4) fractions in a ratio (W/W) of 21:1.0:3.8. The subsequent isolation was performed in an anti-HSV-1 assay-guided way. PVP-2, which had the most potent anti-HSV-1 activity, was further fractionated by gel exclusion chromatography on a column (2.2×90 cm) of Sepharose CL-6B (Pharmacia, Sweden) using 0.2 M NaCl. One fraction near the void volume (PVP-2a) and one fraction near the inner volume (PVP-2b) were obtained (FIG. 8). All the obtained fractions were dialyzed against water (yield ratios (W/W), PVP-2a: PVP-2b=1.5:1.0). These fractions were consequently purified by fractionation on a column (2.2-90 cm) of Sephadex 0-100 (Pharmacia, Sweden). The purified PVP-2a and PVP-2b were assayed for their anti-HSV-1 activities by the plaque reduction assay (see Examples 3 & 6 below). The purified PVP-2b (PVP complex) showed potent antiviral activity with its IC₅₀ of about 18 g/ml and PVP-2a showed no activity.

### Example 3

### Purification of Extract Fraction by Column Chromatography

The active anti-herpes component was further purified by gel filtration column chromatography. An aqueous solution of PVP (450 mg in 10 ml from extraction method 1) was applied to a Sephadex G-50 column (98×2.5 cm) and eluted with water. Fractions of 5 ml were collected. The anti-herpes activity was detected using the plaque reduction assay. To do this, fractions were freeze-dried and redissolved in distilled water to 1 mg/ml. Plaque reduction assay was performed according to the standard method described by Edgar et al. (see Manual of clinical microbiology-5th ed. A. Balows (chief ed.) Amer. Soc. for Microbiology, Washington D.C. (1991), p. 1184-1191). Briefly, monolayers of Veto cells grown on culture plates were infected with 100-200 pfu (plaque-forming unit) of virus. After incubation for 1 h to allow viral adsorption, the inoculum was aspirated and overlaid with medium (Dulbecco's Modified Eagle's medium with 2% fetal calf serum) containing dilutions of the Prunella vulgaris extract in 2% methylcellulose. After 72 h of incubation at 37° C., the plates were fixed with formalin, stained with crystal violet; dh dried and the number of plagues counted. Plates overlaid with medium without the extract were used as controls. The percentage of inhibition of plaque formation was calculated as:

(# of plagues in control)−(# of plagues in test)×100/ (# of plaques in control)]

Active fractions were pooled and freeze-dried. The control plates in which test compound was omitted had an average of 188.5 plaques/well.

The active anti-herpes activity was found in fractions number 91 to number 131 (see Table 1, which presents the anti-HSV-1 activity of fractions from the Sephadex G-50 column). These fractions were pooled to give pooled fractions C, D, E, and F with the highest activity found in fraction E (see FIG. 2). The amount of material recovered in each of the pooled fractions was indicated.

TABLE 1
Anti-HSV-1 activity of fractions from the Sephadex G-50 column

| | % Plaque inhibition | | | |
|---|---|---|---|---|
| Pooled fractions | Fraction No. | 75 μg/ml | 50 μg/ml | 25 μg/ml |
| A (186 mg) | 41 | NTₐ | 0 | 0 |
| | 45 | 0 | 0 | 0 |
| | 51 | 17.8 | 7.2 | 2.9 |
| | 56 | 22.0 | 11.9 | 0 |
| | 61 | 2.4 | 0 | NT |
| | 65 | 11.5 | 2.4 | NT |
| | 71 | 26.8 | 0 | NT |
| B (85 mg) | 75 | 49.1 | 16.2 | 0 |
| | 81 | 79.8 | 56.5 | 21.5 |
| | 85 | 86.7 | 46.9 | 10.3 |
| C (58 mg) | 91 | 100 | 49.6 | 7.7 |
| | 95 | 100 | 100 | 11.9 |
| | 101 | 100 | 100 | 45.9 |
| D (42 mg) | 105 | 100 | 100 | 65.5 |
| | 111 | 100 | 100 | 92.0 |
| | 115 | 100 | 100 | 82.5 |
| E (5 mg) | 121 | 100 | 100 | 84.6 |
| | 125 | 100 | 100 | 100 |
| F (32 mg) | 131 | 100 | 54.9 | 19.4 |
| PVP | | 100 | 73.5 | 36.3 |

^{a}NT = not tested

The results of this example confirm that the purified extract fraction isolated from Prunella vulgaris of the present invention has anti-herpes activity.

### Example 4

### HPLC Analysis of the Purified Extract Fraction

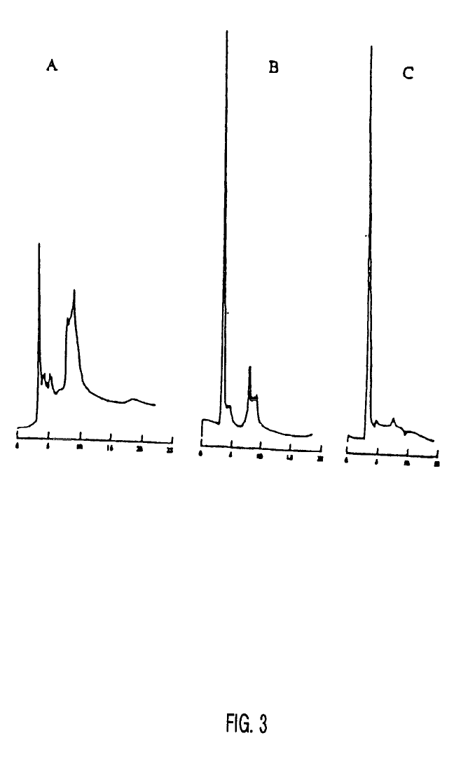

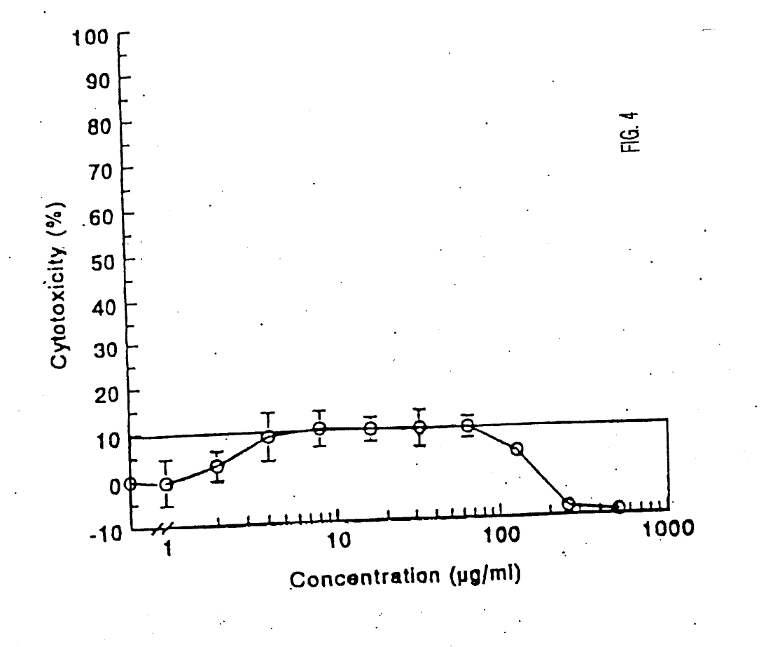

Purity of the anti-herpes materials from Prunella vulgaris was assessed by analyzing three preparations (aqueous extract, PVP, and Fraction E) by reversed-phase high pressure liquid chromatography (HPLC). Aliquots (25 μl) of aqueous solutions (10 μg/ml) were injected into a C18 column (25 cm×4.6 mm ID, 5μ, Supelcosil LC-18, Sigma). Compounds were cluted with 5% water: 95% acetonitrile at a flow rate of 0.3 ml/min. Compounds were detected with a UV detector at 210 nm. The peak with a retention time of 3.56 mm was concentrated during the purification process (see FIG. 3). In Fraction E, this 3.56 mm peak was essentially the only peak. Together with the anti-herpes test results provided in Example 5 (which demonstrate an increase in specific anti-HSV-1 activity during purification), the results of this example confirm that the 3.56 mm peak is the purified extract fraction of Prunella vulgaris, which purified extract fraction has enhanced anti-herpes activity.

### Example 5

### Inhibitory Activity, IC₅₀

Potency of the anti-herpes compound from Prunella vulgaris was assessed by using a range of concentrations of PVP and Fraction E was used to inhibit plaque formation by HSV-1 in Vero cells. The percentage of plaque inhibition was dependent on the amount of PVP and Fraction E added (see Table 2). IC₅₀ is defined as the concentration of extract that causes 50% inhibition in the number of plaques formed in the assay system described. Zero inhibition is where the number of plaques formed, during the assay, is equivalent to or more than the number of plaques formed by control, where the extract is not present. One hundred percent inhibition is where there are no plaques formed during the assay. The concentration of PVP and Fraction E required to give 50% inhibition (IC₅₀) was calculated to be about 18 and 10 μg/ml, respectively. Table 2 presents dose dependent inhibition of plaque formation by PVP and Fraction E (the control which contained no test compound had an average of 64.25 plaques/well).

TABLE 2
Dose dependent inhibition of plaque formation by PVP complex and
fraction E

| PVP | | | |
|---|---|---|---|
| (μg/ml) | % plaque inhibition | Fraction E (μg/ml) | % plaque inhibition |
| 50 | 96.9 | 50 | 100 |
| 25 | 61.1 | 25 | 100 |
| 12.5 | 35.4 | 12.5 | 55.6 |
| 6.25 | 29.2 | 6.25 | 35.4 |
| 3.125 | 0 | 3.125 | 13.6 |

The results of this example confirm that the purified extract fraction of the present invention isolated from Prunella vulgaris has anti-herpes activity.

### Example 6

### Antiviral Activity of Invention Composition Prepared by Method 2

In vitro anti-HSV activity was assayed by plaque reduction method described by Hill et al. (In: Manual of clinical microbiology, 5^{th} ed. American Society for Microbiology, Washington, D.C., p. 1184-1191, 1991).

Cells & Virus: Vero cells (Green Monkey Kidney cells, originally obtained from Professor K. McCarthy at the University of Liverpool, England) used here were grown in Earle's minimum essential medium (MEM-Earle's) at a pH of 7.2 supplemented with 10% heat-inactivated inactiveted fetal calf serum (FCS) Two-three day old confluent monolayer culture were prepared in 6 well culture plate (Falcon, USA) at 37° C. with 5% CO₂ in a humidified incubator. HSV-1 (acyclovir-sensitive strain, BW-S strain, originally obtained from Jack Hill, Borroughs Wellcome Co) was plaque-purified three times under an agarose-overlaid medium. Viral stock was prepared by placing an inoculum of the plaque-purified virus on Vero cell monolayers. Following adsorption for 1 h at 37° C. with 5% CO₂, the virus inoculum was removed and the cells were overlaid with maintenance medium (MM) and incubated at 37° C. with 5% CO₂ for 72 h. The infected culture medium was harvested and centrifuged for 15 min at 900 g. Supernatant was collected, aliquoted in 0.5 ml volumes and stored at −70° C. for use. The virus stock contained 1.1×10^{8laque-forming units (pfu)/ml.}

Plaque Reduction Assay by Extraction Fractions: Confluent Vero cell monolayers were grown in 6-well culture plates and infected with 0.5 ml of MEM Earle's (supplemented with 2% FCS) containing 200 pfu of virus. Viral adsorption was allowed for 1 h at 37° C. in a humidified atomsphere with 5% CO₂ with rocking mannually every 10 min. After removal of inculum, monolayers were overlayed with 4 ml of serial dilutions of the p. vulgaris extract in 0.8% methyleellulose in MM. Controls were parallel prepared on plates overlaid with 0.8% methylcellulose in MM without the dilutions of the p. vulgaris extract. Then the plates were incubated at 37° C. for 72 h followed by fixing with 10% formaldehyde overnight, and staining with 1% crystal violet in 20% ethanol. The number of plaques was counted after air-drying. Results are expressed by percentage of plaque reduction versus untreated controls:

n# of plagues in control)−ean# of plagues in test)×100/ (mean# of plaques in control)]

The anti-HSV-1 IC₅₀ of the crude polysaccharide PVP-1 was found to be about 25 μg/ml. CTAB precipitation led to three subfractions, namely, as acidic (PVP-2), weakly acidic (PVP-3) and neutral (PVP-4). PVP-2 showed the most potent anti-HSV-1 activity with IC₅₀ of about 22 μg/ml whereas PVP-4 showed no anti-HSV-1 activity. PVP-2 was fractionated on Sepharose CL-6B column to obtain two subfractions (PVP-2a and PVP-2b). The subfractions were subsequently purified on Sephadex G-100 column and assayed for their anti-HSV-1 activities by plaque reduction assay. The subfraction near the inner volume (PVP-2b) showed significant anti-HSV activity with an IC₅₀ of about 18 μg/ml. The fraction near the void volume (PVP-2a), which was demonstrated to be composed mainly of polysaccharides, did not show any anti-HSV-1 activity. These results indicate that PVP-2b is the active ingredients in PVP-2 to account for the antiviral activity of P. vulgaris.

### Example 7

### Spectrum of Activity

A number of viruses were used in the plaque reduction assay to assess the spectrum of activity of the extract fraction from Prunella vulgaris. Results showed that PVP at 100 μg/ml provided complete inhibition of plaque formation in Vero cells by laboratory and clinical strains of HSV-1 and HSV-2. PVP, at 100 μg/ml, also inhibited acyclovir-resistant strains of HSV-1 [strain DM2-1 (thymidine kinase-deficient) and strain PAAr5 (DNA polymerase-deficient)] and HSV-2 strain Kost (thymidine kinase altered). PVP at 100 μg/ml showed no activity against cytomegalovirus, human influenza virus types A and B, poliovirus type 1, and vesicular stomatitis virus. For the test with cytomegalovirus and human influenza viruses, human foreskin cells and MDCK (dog kidney) cells, respectively, were used in place of Vero cells.

The results of this example confirm that the extract fraction from Prunella vulgarishas specific activity against HSV-1 and HSV-2 and further demonstrates that since it is active against acyclovir-resistant HSV, the extract fraction from Prunella vulgaris can be a useful drug of choice in treating the cytopathogenic effects of infections caused by acyclovir-resistant HSV.

### Example 8

### Effects of P-prunella vulgaris Anti-Herpes Compound on HSV-1 Infection

The mode of action of the anti-herpes effect of PVP complex was studied. Vero cells were preincubated with 75 μg/ml PVP complex for 16 to 20 h at 37° C. Cells were washed with medium and infected with HSV-1. The same number of plaques (50 plaques/well) was observed as that in controls in which cells had not been treated with PVP complex. This indicates that preincubation of Vero cells with PVP complex has no protective effect.

To study whether preincubation of virus with PVP complex will abolish infectivity, 100 μg of PVP complex was incubated with 10⁵ pfu of HSV-1 at 37° C. After 1 h incubation, the mixture was diluted 10,000 fold with medium and used to infect Vero cells. No plaque was observed on the monolayers after incubation. In contrast, the control plate, in which the viruses were pretreated with medium instead of PVP complex, contained an average of 130 plagues/well. This finding indicates that the in fectivity of HSV-1 was abolished by preincubation with PVP complex, which probably exerts its effect by binding to the viral particles and preventing them to bind to heparan sulfate on Vero cells. This mode of action to reduce infectivity of herpes is completely different from acyclovir and other known nucleoside analogs.

The protective effect of PVP complex was demonstrated by adding PVP complex simultaneously with HSV-1 to Vera cells. When 75 μg/ml of PVP complex was added at the same time with HSV-1 to Vera cells, greater than 98% reduction in plaque formation was observed. Incubation of 100 μg PVP complex and 106 plaque-forming units of HSV at 4° C., ambient temperature (25° C.) and 36° C. for 1 h abrogated 99% of the virus infectivity. The protective effect of PVP complex was further demonstrated when PVP complex was added post infection.

The effect of PVP complex on HSV-1 growth in Vero cells was further investigated in a one-step growth study. Monolayers of Vera cells were infected, at 4° C., with HSV-1 at a multiplicity of infection (MOI) of 5, i.e. 5 virions per cell. At this temperature, the virus would bind but would not penetrate the cells. Hence, all cells in the monolayer were synchronized at the same step of viral infection. The cells were washed with cold medium and treated with 75 μg/ml PVP complex at 0, 2, 4, and 7.25 h after the washing step. Total viral yield at each time point was determined by plating out samples from the supernatant (extracellular) and lyzed cells (intracellular). Results confirm that when PVP complex was added at 0, 2, 4, and 7.25 h after infection, the total viral yield was reduced by 99, 96, 94, and 90%, respectively, as compared to controls where cells were not treated with PVP complex.

The results of this example confirm that the extract fraction from Prunella vulgarisalso acts on herpes virus intracellularly to reduce the yield of infectious virus and prevents cell-to-cell transmission of the virus, and that the extract fraction interferes intracellularly with certain biosynthetic steps in the viral replication process.

### Example 9

### Chemical Nature of Anti-Herpes Compound from prunella vulgaris

The chemical nature of the anti-herpes compound was investigated by different chemical tests. Total carbohydrate content was estimated by the phenol sulfuric acid assay using glucuronic acid as the standard, which results showed the anti-herpes compound in Fraction E contained about 42% (w/w) carbohydrates (expressed as glucuronic acid). Uronic acids were measured with the method described by Blumenkrantz and Asboe-Hansen (see Anal. Biocehm. 54: 484-489 (1973)) using glucuronic acid as the standard. The anti-herpes compound contains about 7.5% (w/w) uronic acid (expressed as glucuronic acid). Total hexosamines were determined by the Molgan-Elson reagent (see Whiteman, P. in Biochem. J. 131: 343-350 (1973)) using N-acetyiglucosamine (Sigma) as the standard. Hexosamines were not detected. Protein was measured by the Coomassie Blue dye binding method (Bio-Rad) using bovine serum albumin as the standard. Protein has not been detected. Elemental analysis showed the purified extract fraction to contain 31-35% carbon, preferably 31%, more preferably 30.78%; 3-4% hydrogen, preferably 3.1%, more preferably 3.05; 0.5-1.0% nitrogen, preferably 0.7%, more preferably 0.66%; and 2-3% sulfur, preferably 2.7%, more preferably 2.69%.

The anti-herpes compound was found to be precipitated by the cationic dye Alcian blue 8GX according to the assay method described by Whiteman (see Biochem. J. 131: 343-350 (1973)). The anti-herpes compound bound strongly to DEAE Sepharose at neutral pH and could be eluted with 2 M NaCl. These experiments confirm the anti-herpes extract fraction from Prunella vulgaris contains a polyanionic carbohydrate.

The anti-herpes compound was water soluble, but was insoluble in methanol, ethanol, butanol, acetone, or chloroform. The compound is heat stable (95-100° C., 4 h). A 1 mg/ml aqueous solution of the anti-herpes compound gave a pH of 5.5. Spectrophotometry showed a strong absorption peak at 202 nm and a shoulder at 280 nm which extended to 380 nm. In contrast, the prunellin previously isolated by Tabba et al (see Antiviral Res. 11: 263-274 (1989)) has a pH of 7.4 in aqueous solution, and an absorption peak at 370 nm which extended to 500 nm.

The results of this example confirm that the anti-herpes extract fraction from nella vulgaris different from prunellin.

### Example 10

### Active Constituents Analysis: Protease Digestion and Periodate Oxidation

For determining the antiviral contribution of possible protein moieties in the active constituents, a protease digestion procedure was used to decompose the protein moieties. This procedure was performed as described previously by Zhang et al (Planta Med. 63: 393-399, 1997). PVP-2b (25 mg) was digested with protease (6.3 mg, 9 units/mg, Sigma) in 50 mM Tris-HCl buffer (pH 7.9) containing 10 mM CaCl₂ (40 ml) at 37° C. for 96 h. The reaction was terminated by neutralization with 0.1 M HCl, and then the mixture was dialyzed against water and lyophilized to obtain the protease digested product (PVP-2b-PR, yield: 86.0%).

For determining the antiviral contribution of carbohydrate moieties in the active constituents, a controlled periodate oxidation procedure was used to decompose the carbohydrate moieties. The procedure was performed as described previously by Zhang et al (Planta Med. 63: 393-399, 1997). PVP-2b (20 mg) was oxidized with 50 mM NaIO₄ in 50 mM acetate buffer (pH 4.5) (40 ml) at 4° C. for 96 h in the dark. After the reaction had been terminated with ethylene glycol, the product was reduced with NaBH₄ (40 mg) and dialyzed to obtain periodate oxidized product (PVP-2b-SD, yield: 52.8%).

The crude polysaccharide fraction PVP-1 together with the CTAB precipitation subfractions (PVP-2, 3 and 4) were compared in their general chemical properties. The percentages of carbohydrates, and uronic acids were calculated based on the absorptions of the samples and equations of respective correlation curves of galactose, and galacturouic acid (Table 3).

TABLE 3
General properties and the anti-HSV-1 activities of the crude
polysaccharide fraction from P. vulgaris

| | PVP-1 | PVP-2 | PVP-3 | PVP-4 |
|---|---|---|---|---|
| Neural sugar^{a} (%) | 52.5 | 62.1 | 85.2 | 98.7 |
| Uronic acid^{a} (%) | 25.7 | 22.8 | 8.2 | 3.7 |
| IC₅₀ (g/ml)^{a} | 25 | 22 | 24 | ND^{b} |

^{a}Average of two determinations.
^{b}ND, not determined.

The subfractions (PVP-2a and 2b) of PVP-2 on gel exclusion chromatography on Sepharose CL-6B were analyzed for their composing sugars besides the general chemical properties. They were also showed as single peaks by fractionation on Sephadex G-100. When they were analyzed by HPLC on Shodex sugar KS-850+KS-840 column, PVP-2a and PVP-2b were eluted as single peaks having respective molecular weights of about 93 kDa and about 8.5 kDa (Table 4). PVP-2b was found to consist of carbohydrate (12.2%) with arabinose, xylose, rhamnose, mannose, galactose, glucose and galacturonic acid in a molar ratio of 0.1:0.3:0.3:0.7:1.0:3.4:0.5. On the other hand, PVP-2a was found to consist of carbohydrate (98.7%) with arabinose, xylose, rhaninose, galactose, glucose and galacturonic acid in a molar ratio of 4.0:11.1:0.1:1.0:4.0:1.1 as well as minor mannose. When PVP-2b was analyzed for protein content by the Bradford method, it seemed to contain large amounts of protein-like substances from the Bradford-positive results (Table 4). However, when PVP-2b was analyzed by elemental analysis, only about 3.8% of nitrogen was found. The results suggest that PVP-2b contains no protein.

TABLE 4
Chemical properties of anti-HSV-1 fraction PVP-2 and its subfractions
from P. vulgaris

| | PVP-2 | PVP-2a | PVP-2b |
|---|---|---|---|
| Molecular weight | − | 93,000 | 8,500 |
| Neutral sugar (%) | 62.1 | 98.7 | 12.2 |
| Uronic acid (%) | 22.8 | 26.2 | 6.6 |
| Protein^{a} (%) | 17.4 | − | 17.4 |
| Nitrogen^{b} (%) | | n.d.^{c} | 3.8 |
| Lignin (%) | 13.6 | n.d. | 24.4 |
| Vanillin | + | − | + |
| Syringaldehyde | + | − | + |
| p-Hydroxybenzenaldehyde | + | − | + |
| Component sugar (Mol. %) | | | |
| Ara | − | 4.0 | 0.1 |
| Xyl | − | 11.1 | 0.3 |
| Rha | − | 0.1 | 0.3 |
| Man | − | n.d. | 0.7 |
| Gal | − | 1.0 | 1.0 |
| Glc | − | 4.0 | 3.4 |
| GalA | − | 1.1 | 0.5 |

^{a}Data obtained from Bradford method.
^{b}Data obtained from elemental analysis.
^{c}n.d., not determined

In addition, UV spectrum of PVP-2b showed similar absorption maximums at 245, 275 and 312 nm. Compared with the spectra of commercially available substances, PVP-2b showed containing polyphenolic compound, lignin. PVP-2b was then subjected to alkaline nitrobenzene oxidation in order to analyze the presence of lignin molecule, and the resulting products were analyzed by (GLC-MS. The oxidation derivatives, vanillin, syringaldehyde, and p-hydroxybenzaldehyde were detected in the oxidation products derived from PVP-2b (Table 4), indicating that PVP-2b comprised lignin moieties in addition to carbohydrate moieties. When lignin-content in PVP-2b was calorimetrically measured by the acetyl bromide method (Dence, 1992), PVP-2b was estimated to contain about 24.4% of lignin (Table 4). Therefore, all evidence of chemical properties of PVP-2b support the fact it is a lignin-carbohydrate complex (PVP complex).

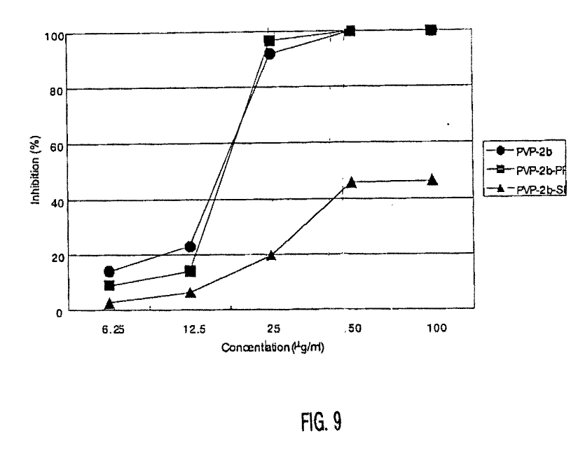

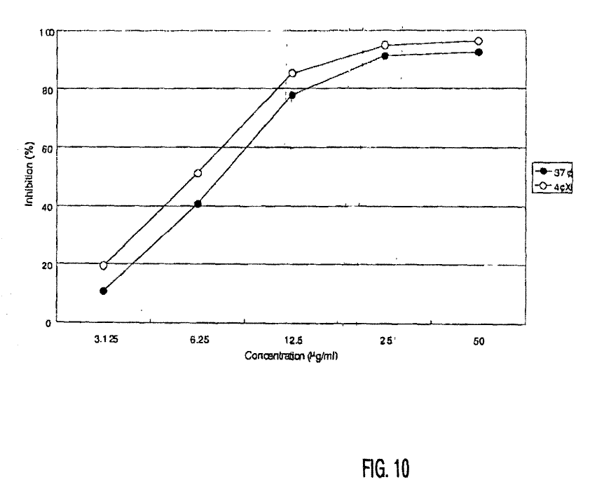

PVP-2b was treated with NaIO₄ to decompose the carbohydrate moieties in its molecules and the periodate oxidized product PVP-2b-SD was obtained with a yield ratio of about 52.8%. It was also treated with protease to decompose the possible protein moieties and the protease digested product PVP-2b-PR was obtained with a yield ratio of about 86.0%. The samples were assayed for their anti-HSV-1 activities at the final concentrations of 100, 50, 25, 12.5 and 6.25 μg/ml, by plaque reduction method. The IC₅₀ of PVP-2b-PR was found to be about 18 μg/ml and IC₅₀ of PVP-2b-SD was found to be over 100 μg/ml. Treatment with periodate oxidation will reduce the anti-HSV-1 activity of PVP-2b significantly and treatment with protease digestion did not affect the activity through the antiviral assay of PVP-2b-SD and PVP-2b-PR (FIG. 9). It was therefore assumed that a combination of lignin moiety and the carbohydrate moiety in PVP-2b might be responsible for the antiviral activity. Heparin (Sigma, USA) was used in this assay at the final concentrations of 1000, 500, 250, 125 and 62.5 μg/ml as positive control and its IC₅₀ was found to be 300 μg/ml.

### Example 11

### Cytotoxicity

The cytotoxic effect of the aqueous extract on mammalian cells was tested using a rat intestinal epithelial cell line (RIE 1) according to a published method (see Blay, J., and A. S. L. Poon in Toxicon. 33: 739-746 (1995)). Other cell lines including those originating from humans, such as T84 human intestinal epithelioid cells and KB human oral epidermoid cells can also be tested to ensure the lack of toxicity is not limited to one cell line or species. The dried aqueous extract was dissolved in DMSO and diluted in culture medium (Dulbecco's Modified Eagle's medium with 5% (v/v) heat-inactivated calf serum) before added directly to RIE-1 cells. The cultures were incubated for 48 h. MTT (3-{4,5-dimethylthiazol-2-yl]-2,5-diphenyl tetrazolium bromide) was added to the culture wells to give a final concentration of 0.5 mg/ml. The cultures were incubated for 3 h at 37° C. to allow the conversion of MTT to formazan dye by mitochondrial succinate dehydrogenase. The dye was measured at A₄₉₂ with a Titertek Multiscan plate reader. Plates containing the same amount of DMSO, but without the test extract were used as controls. The percent cytotoxicity was calculated by comparing the A₄₉₂ readings from the tests relative to A₄₉₂ readings from control wells in which the extract fraction was omitted.

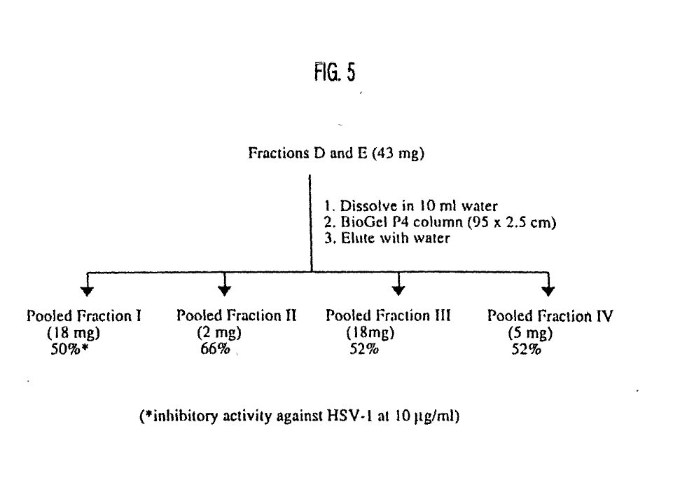

The results of this example confirm that the aqueous extract fraction of Prunella vulgaris shows no cytotoxic effect up to the highest concentration tested, i.e., 500 μg/ml (see FIG. 4).

The series of experiments described in the above document that invention compositions obtained from Prunella vulgaris are lignin-carbohydrate complex (PVP complex), which are nontoxic up to 500 μg/ml, and have specific novel activity against enveloped viruses, and specifically, strains of HSV-1, HSV-2, including acyclovir-resistant strains.

The active extract fraction from Prunella vulgaris can be purified to homogeneity and its chemical and biological characteristics determined in order to understand its chemical nature, mode of action, and spectrum of activity, and thereby used to improve its utility and potency as an antiviral drug, according to the following examples.

### Example 12

### Isolation and Purification of Anti-Herpes Extract Fraction from Prunella vulgaris

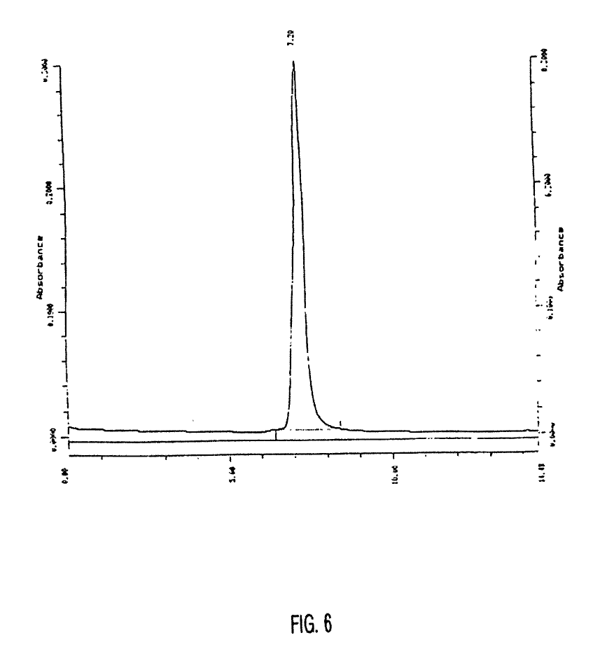

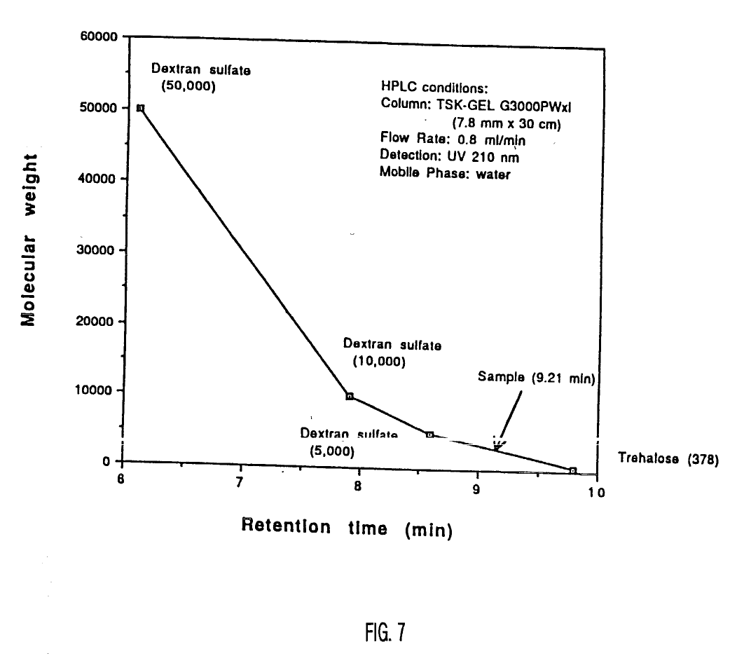

The anti-herpes anionic polysaccharide complex was purified by re-chromatographing pooled materials from Fractions D and E (see Example 3) on a BioGel P4 column (95×2.5 cm) using methods as described. The yield and inhibitory activity against HSV-1 of fractions obtained are shown in FIG. 5. The purified extract fraction of the present invention can also be prepared by extraction, precipitation, and gel filtration chromatography, as disclosed herein. Alternative means for purification, for example, density equilibrium centrifugation, ultrafiltration, and dialysis can also be employed. More economical purification of the active compound for industrial scale processes can be achieved by, for example, ionic interaction chromatography (DEAE-Sepharose 4B) and reversed-phase high pressure liquid chromatography. Since the active compound binds strongly to DEAE Sepharose 4B at neutral pH and can be eluted with 2 M NaCl, the ethanol-precipitated compounds can be applied to a DEAE-Sepharose column, the active compound eluted with a NaCl gradient, and the active fractions identified by standard plaque reduction assays. Thereafter, the active fractions can be pooled, dialyzed, and further purified by HPLC with a preparative reversed-phase (C-18) column, a representative purification process which can scaled up on industrial scale HPLCs. When a sample from fraction IV was analyzed by HPLC, a single peak was obtained, indicating the anti-herpes compound is pure (FIG. 6). The conditions for the HPLC were as follows: column—TSK G3000 PW×1, 7.8 mm×30 cm, 6 μm; flow rate of 0.8 ml/min; mobil phase-water; detection—UV 210 nm; injection of 20 μl and a concentration of 0.2 mg/ml.

### Example 13

### Chemical Characterization of Anti-Herpes Compound from P. vulgaris

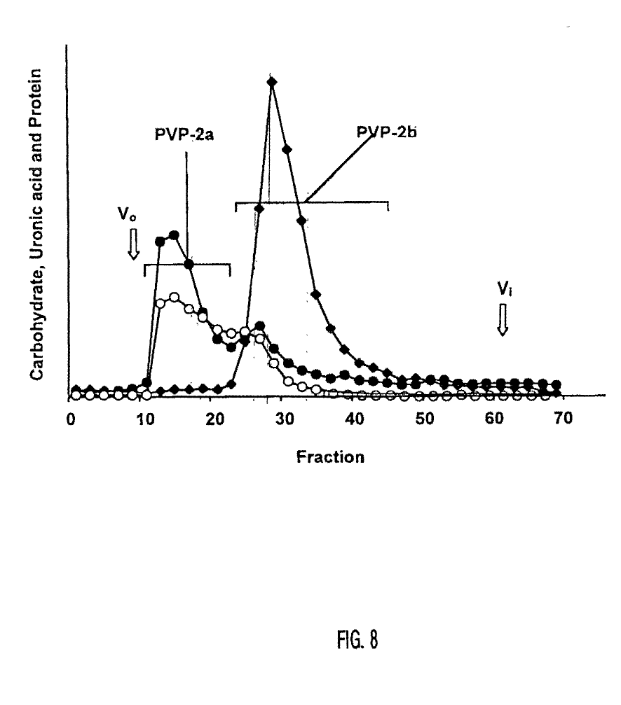

The next objective was to determine the molecular weight or mass of the purified compound. The most commonly employed methods to determine molecular mass of macromolecules are gel permeation chromatography, HPLC exclusion chromatography, osmotic pressure, SDS gel electrophoresis, the Squire method using G-75, dialysis through membranes with selected molecular mass cut-offs, ultracentrifugation with sedimentation rate measurement, and the like. The molecular mass of the purified compound was estimated by HPLC with a gel filtration column (TSK-GEL G3000 PW×1, 7.8 mm×30 cm, 6 μm). The purified compound has an estimated molecular mass of 3,500 kDa. (FIG. 7; This is in contrast to the prunellin previously isolated by Tabba et al.(Antiviral Res. 11(5-6): 263-273, 1989) which has a molecular mass of 10,000 kDa). Elemental, Infrared, NMR and other spectroscopic analytical means can also be employed to characterize the active compound.

The purified compound was hydrolyzed in 2 N trifluoroacetic acid at 121° C. for 1 h. When the hydrolysate was analyzed by paper chromatography (solvent system: pyridine:ethylacetate:water=4:10:3), three spots which have the same R_{f} values of glucose, galactose and xylose standards were obtained. The product can also be exhaustively hydrolyzed in acid (e.g., 2N HCl) and analyzed for constituent monosacharides by HPLC, and gas chromatography. By comparing the intensity of the spots, glucose was the major constituent sugar. Galactose and xylose were minor components. These data were consistent with those reported in Table 4.

These results indicate that the purified anti-herpes polysaccharide in PVP complex is composed of mainly glucose with some galactose and xylose as the constituent monosaccharides. Some of these monosaccharides are likely to have SO₄ and COOH groups on them to confer the anionic nature of the polysaccharide. Known assays, such as uronic acid assay (see Blumenkrantz, N. and G. Asboe-Hansen in Anal. Biocehm. 54: 484-489 (1973)) and Molgan-Elson assay (see Ghuysen, J. M., et al., in Methods Enzymology 8: 685-699 (1966)), can be employed to verify the amounts of uronic acid and hexosamines present. Other characteristics of the isolated compounds, for example, pI, pH of aqueous solution, and solubility, can be determined by means known in the art.

### Example 14

### Biological Characterization of Anti-Herpes Compound from P. vulgaris

1. Activity of the Purified Polysaccahride Complex on HSV

As disclosed herein, the semi-purified extract fraction has two mechanisms of anti-HSV activity in vitro by acting extracellularly and intracellularly against the virus. Since the active compound(s) binds extracellularly, thereby reducing the yield of infectious virus and preventing cell-to-cell transmission of the virus, it is likely the compound interferes intracellularly with other functional or biosynthetic steps in the viral replication process.

Specific modes of action can be elucidated by using means known in the art, for example, by the virion binding assay. In this example, ³⁵S-labeled HSV-1 is purified by sucrose gradient (20-60%, w/w) fractionation of culture fluids from 7-20 h virus-infected Vero cells grown in methionine-deficient medium containing 2% dialyzed fetal bovine serum and ³⁵S-methionine (10 μCi/ml, specific activity >800 Ci/moles, New England Nuclear). The radiolabeled virus is incubated with the purified compound or medium at 4° C. and then added to monolayers of Vero cells at a multiplicity of infection (MOI) of 1. Following 1 h incubation at 4° C. to allow virus adsorption, cells are washed free of unadsorbed viruses with phosphate-buffered saline containing bovine serum albumin (0.5%). The extent of virus binding can be compared by measuring the radioactivity in monolayers infected with compound treated or medium-treated virus. Alternatively, monolayer cells can be solubilized with a detergent, and the cell-bound radiolabel analyzed by SDS-PAGE and fluorography.

The major viral capsid protein VP5 can be used as a convenient protein for densitometric quantitation (see Herold, B. C., et al., J. Virol. 65: 1090-1098, 1991). Other binding experiments known in the art can be employed to study the effect on invention compound on virus binding kinetics, and on virus already bound to Vero cells.

The specific role of the Prunella vulgaris compound in virus binding in relationship to gC and gB can be further validated using gC-deficient and gB-deficient mutants of HSV-1 and specific anti-gC and gB antibodies. The effect of the compound on virus penetration can be studied using known methods in the art, for example, Herold et al., J. Virol. 22: 3461-3469, 1996), which determines the resistancy of an adsorbed virus to a pH 3.0 buffer.

Intracellular anti-HSV activity of the compound can be studied by infecting Vero cells with HSV-1 at a MOI of 20 at 4° C., and subsequent virus growth (from extracellular supernatants and cell lysates) in the presence or absence of the compound then compared at 0, 0.5, 1, 2, 4, 8, 12, 16, and 20 h post-infection at 37° C. This example of a “single-cell growth” experiment is expected to confirm the invention results disclosed herein, specifically, that the addition of the compound following infection reduces virus yields in cultures. Ultrastructural studies on these samples can be performed using known methods (see, for example, Gollins and Porterfield, J. Gen. Virol. 66: 1969-1982 (1985). Comparison of the mode of entry of HSV-1 into cells, with the subsequent morphological development in the cellular and subcellular compartments of infected cells incubated in the presence or absence of the compound, will lead to increased knowledge of the fundamental viral inhibitory characteristics of invention compound and can be used to define the mode of action of invention compound at the biochemical and molecular level.

TABLE 5
Activity of the Purified Polysaccharide on HSV

| | | | % Plaque Inhibition | | | |
|---|---|---|---|---|---|---|
| | Acyclovir | | 50 | 25 | 12.5 | 6.25 |
| Virus | sensitivity | Strain #^{a} | μg/ml^{b} | μg/ml | μg/ml | μg/ml |
| HSV-1 | Sensitive | 15577 | 100 | 95 | 49 | 0 |
| HSV-1 | Resistant | 12959 | 100 | 100 | 14 | 0 |
| | Thymidine | | | | | |
| | kinase | | | | | |
| | deficient | | | | | |
| HSV-1 | Resistant | 15518 | 100 | 88 | 20 | 0 |
| | Thymide | | | | | |
| | kinase | | | | | |
| | altered | | | | | |
| HSV-2 | Sensitive | 15614 | 100 | 100 | 41 | 18 |
| HSV-2 | Resistant | 15597 | 100 | 95 | 49 | 0 |
| | Thymidine | | | | | |
| | kinase | | | | | |
| | altered | | | | | |

^{a}Well-characterized strains from Burroughs Wellcome Co.
^{b}Concentrations of the purified anti-herpes compound used.

These results show that the purified polysaccharide complex from P. vulgaris contains activity against both types of herpes viruses regardless of whether they are sensitive or resistant to acyclovir. The results indicate that the PVP complex has a different mode of action than acyclovir and imply that it can be used for treatment of infections caused by acyclovir-resistant herpes viruses.

The activity of the purified compound of the extract fraction can be tested against viruses in the herpes family (e.g. VZV and cytomegalovirus) and other enveloped viruses, such as human immunodeficiency virus type 1 (HIV-1), human cytomegalovirus, virus, and the like, to determine its full spectrum of activity and anti-coagulant activity, useful for determining its specific utility for a given enveloped virus.

2. Anti-Coagulant Activity

The anti-coagulant activity of the PVP complex was measured by the prothrombin time test. The prothrombin times were measured at 37° C. using a BBL fibrometer (Becton Dickinson and Co, USA). Blood (9 ml), collected from the rabbit marginal ear vein, was mixed with 3.8% sodium citrate (1 ml) and centrifuged at 1,500×g for 10 min at room temperature to obtain a clear supernatant as the testing plasma. In a typical assay, the mixture containing 50 μl of the testing solution (1 mg/ml), 150 μl of 50 mM Tris buffer, 0.1 M HCl, pH 7.5, 100 μl of thromboplastin with calcium and 100 μL of plasma, was incubated at 37° C. The prothrombin. times were recorded in seconds as the fibrometer stopped due to clotting.

The average prothrombin time for the anti-herpes PVP complex was 25.9±1.5 seconds, a value similar to the water control 29.9±1.4 seconds. Water is employed as a control to show the normal prothrombin time of the plasma. The results showed that anti-herpes PVP complex has substantially little or no anti-coagulant activity. This is in contrast to a known anti-coagulant anionic polysaccharide, heparin, which has also been described to have anti-HSV activity (Herold et al., J. Virol. 22: 3461-3469, 1996). The probrombin time for anti-coagulant (e.g., heparin) is about 300 seconds.

3. Comparative Studies of the Anti-Herpes Activity Between the P. vulgaris Compound and Sodium Heparin.

The anti-herpes activity of the PVP complex and sodium heparin was studied to illustrate the differences between the two. First, using the plaque reduction assay as described in Examples 3 & 6, a significant difference in the ability to inhibit HSV-1 strain #15577 and strain Delta gC2-3 (a glycoprotein C deficient HSV-1, Herold et al., J. Gen. Virol. 75: 1211-1222, 1994)) was observed. Glycoprotein C is one of the viral envelop proteins that mediates virus binding to heparin receptors on mammalian cells. Heparin is thought to inhibit HSV-1 infection of cells by competing with heparin receptors (Harold et al., J. Gen. Virol. 75: 1211-1222, 1994)). The IC₅₀ of heparin against strains 15577 and Delta gC2-3 were estimated as 750 μg/ml and >1 mg/ml, respectively. In contrast, the IC₅₀ of the PVP complex against strains #15577 and Delta gC2-3 were estimated as 10 μg/ml and 5 μg/ml, respectively.

A second major difference between the PVP complex and sodium heparin was observed by the binding experiment. In this experiment, 0.1 ml of HSV-1 (10⁵ pfu) was incubated with 0.1 ml of sodium heparin (20 mg/ml), or the PVP complex (1 mg/ml), or water at 36° C. for 1 h. The mixture was serially diluted in medium and the number of residual infectious virus was determined by the plaque assay. The number of plaque developed in the water treatment control was taken as 100%. The results showed that after the treatment with 2 mg of heparin, 30% and 42% of the original HSV-1 strains #15577 and Delta gC2-3, respectively, were still infectious and recovered by the plaque assay. In contrast, the same amount of virus after exposure to 100 μg of the PVP complex, none of the virus from both strains remained infectious and could not be recovered by the plaque assay.

The anti-herpes effect of sodium heparin was further investigated by a binding competition experiment. In this experiment, 0.5 ml samples of HSV-1 (about 100 pfu) were mixed with 0.5 ml of sodium heparin at different concentrations. The mixtures were immediately used to infect Vero cells at 37° C. for 1 h. Following the incubation, the number of plaques were determined in the plaque assay. The results showed that there was a 88% inhibition of plaque formation for strain #15577 when 62.5 μg/ml heparin was used. However, complete inhibition of plaque formation could not be achieved even when 1 mg/ml heparin was used. At 1 mg/ml heparin, the % plaque inhibition was 92%. Similar results were obtained when the gC-deficient HSV-1, Delta gC2-3, was used. In which case, the % plaque inhibition for 62.5 μg/ml and 1 mg/ml heparin were 77% and 86%, respectively.

These results suggest that sodium heparin has anti-herpes activity only when it is present at the start of the virus infection. In other words, heparin can have inhibitory effect on HSV only when the virus has not yet bound to the cells to initiate the infection cycle. This statement is further supported by the high IC₅₀ values (750-1000 μg/ml) as determined by the plaque reduction assay. In the plaque reduction assay, heparin was added 1 h after the virus has incubated (i.e. has bound and probably penetrated the cells) with the vero cells. Heparin has no inhibitory effect as indicated by the high IC₅₀ values. The anionic polysaccharide heparin exerts its anti-herpes effect by blocking virus binding to the cells. This blocking can only be effective when the virus has not bound to the cell receptors. This is contrast to the P. vulgaris anionic polysaccharide complex that it can inhibit HSV infection before virus binding as well as after virus binding and penetration. The results described in the one-step growth study (Example 8) clearly show that even after 7.25 h post virus infection, the P. vulgaris polysaccharide complex could reduce virus yield by 90%. In addition, the results described in this example, specifically in the plaque reduction assay and the binding experiment, clearly show that the P. vulgaris polysaccharide complex is a superior anti-herpes agent relative to heparin.

### Example 15

### Virucidal Effects of PVP Complex

Virucidal effect assay was adapted from the method described previously by Xu et al. (Antiviral Res. 44: 43-54, 1999). 0.1 ml of HSV-1 containing 4.4×10⁶ pfu was incubated with 0.1 ml of tested sample in serum-free MEM (pH 7.4) at 37° C. for 1 h. Controls were made by mixing 0.1 ml of HSV-1 with 0.1 ml of medium. The treated virus was promptly 10⁵-fold diluted with MEM supplemented with FCS and assayed for infectivity by plaque reduction method on 6-well plate.

Virusidal effect was assayed by direct incubation of HSV-1 with tested sample. The virus was diluted after pretreatment with the sample and assayed for infectivity by plaque reduction method. In PVP-2b group, it is provided that the 85% of the viruses were inhibited in comparison with the medium treated control, suggesting that PVP-2b has the direct inhibition effect to HSV-1.

### Example 16

### Assays for Effect of PVP Complex on Virus Binding to Vero Cells

HSV-1 stock was diluted to 100 pfu in prechilled MM and mixed with an equal volume of prechilled MM containing 50, 25, 12.5, 6.25 and 3.125 μg/ml of the sample or with only MM for control. The mixtures in 250 μl were immediately inoculated on Vero cell cultures at 4° C. and 37° C. After adsorption for 1 h, the inoeula were removed from the culture followed by washing twice with the medium. Plaque formation was allowed by incubation at 37° C. for 72 h in 0.6% agrose medium.

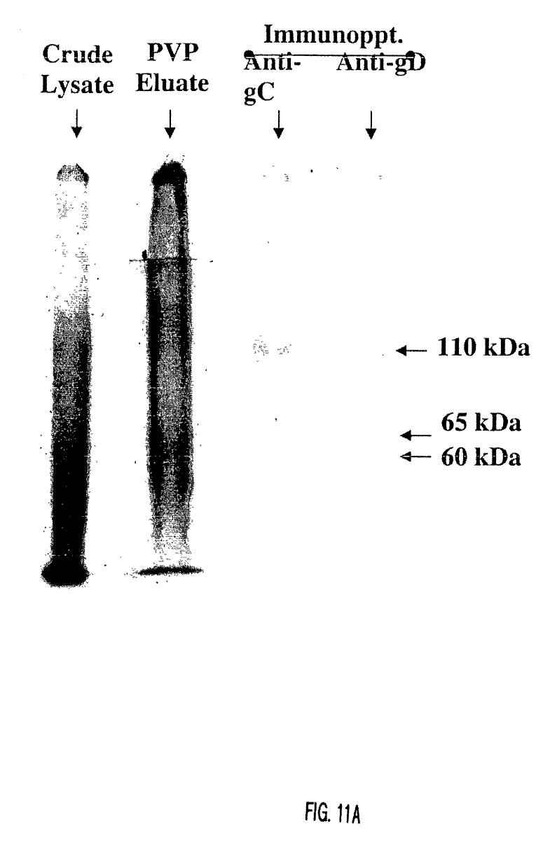

Effect of PVP-2b on virus adsorption to Vero cells was investigated at concentrations of 50 μg/ml to 3.125 μg/ml at 4° C. and 37° C. A dose-dependent impeding effect to virus adsorption to Vero cells was observed at 4° C. However, a similar trend of dose-dependent impeding effect to virus adsorption was also observed at 37° C. In this assay, PVP-2b showed the significant blocking effect to HSV-1 adsorption to Vero cells at 4° C. and 37° C. after pretreating the virus with the sample. The IC₅₀ were found to be 7.4 μg/ml at 37° C. and 6.0 μg/ml at 4° C. Effects of PVP-2b on virus adsorption to Vero cells were not found at 3.125 μg/ml (FIG. 10).

### Example 17

### Effects of PVP Complex on Penetration of HSV-1 into Vero Cells

Penetration Assay. The penetration assay was conducted using the method as described in Example with modifications. Confluent Vero cell monolayers in 25 ml tissue culture flasks were chilled to 4° C. for 45 mm. The monolayers were infected with 300 pfu of HSV-1 in 2.5 ml of cold MEM Earle's medium. Attachment was synchronized for 1 h at 4° C. The flasks were washed twice with 4 ml of cold PBS to remove any unbound virus. The monolayers were covered with 5 ml of maintenance medium and shifted to 37° C. At set time intervals, the medium was removed and the cells were treated one of the following for ways: (a) PVP complex, (b) heparin, (c) citrate buffer, pH 3 (80 mM ciric acid, 40 mM Na₂HPO₄) and no treatment control. For the PVP- or heparin-treated groups, 5 ml of fresh maintenance medium containing 100 μg/ml PVP complex or 1000 μg/ml heparin was added to the monolayers. The flasks were incubated at 37° C. for 90 min, after which the monolayers were washed twice with 4 ml of PBS and covered with 10 ml of 0.8% methylcellulose in maintenance medium. Plaque formation was allowed at 37° C. for 72 h. For the citrate buffer treated group, 3 ml of buffer was added to the monolayers for 1 min. The cells were washed twice with PBS, after which medium was added to allow plaque formation. For the no treatment control group, following medium removal, the monolayers were washed twice with PBS and medium added to allow plaque formation.

Effects of PVP complex on HSV penetration into Vero cells. Tables 6 and 7 showed the effect of PVP on wild-type HSV-1 and gC-deficient HSV-1 mutant penetration into Vero cell. Citrate buffer at pH 3 represents the penetration kinetics of HSV into Vero cells. The low pH removes adherent virus particles from the cell surface (i.e., those that have not yet penetrated). At time 20 min, all the wild type virus particles have penetrated and can not be removed by the low pH buffer (Table 6). When the cultures were treated with PVP complex, a reduction in plaque formation with respect to time of PVP complex addition was observed. This reduction is less obvious than the heparin treatment. At time 0 min, only 19% of the virus could penetrate and form plaque as compared to 49% in the heparin treatment.

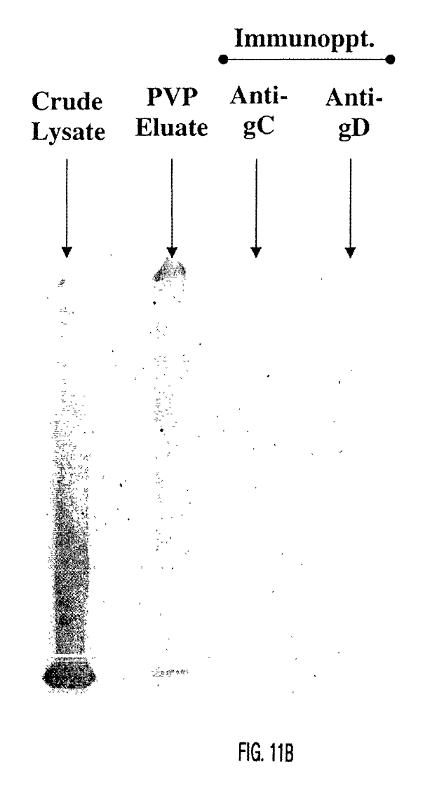

In the case of gC-deficient mutant, the rate of penetration was slower than that displayed by the wild-type HSV (Table 7). However, by 60 min, all the virus particles appeared to have penetrated. The prevention of penetration this mutant by PVP complex was again observed. In contrast, heparin had no apparent effects on the penetration of the gC-deficient mutant into Vero cells. The data indicates that PVP complex can prevent penetration of wild-type HSV-1 better than heparin. The effect is even more clear in the case of the gC-deficient mutant and this suggests that PVP complex acts on gD viral protein in addition to gC (FIG. 11).

TABLE 6
Effect of PVP complex and heparin on HSV-1 penetration into vero cells

| | % Plaques | | | |
|---|---|---|---|---|
| | PVP | Heparin | Citrate buffer, | No treatment |
| Time (min) | (100 μg/ml) | (1000 μg/ml) | (pH 3) | control |
| 0 | 19 | 49 | 0 | 96 |
| 2 | 38 | 57 | 12 | 92 |
| 4 | 48 | 88 | 32 | 100 |
| 6 | 53 | 86 | 50 | 100 |
| 10 | 80 | 100 | 78 | 91 |
| 15 | 95 | 100 | 82 | 95 |
| 20 | 100 | 100 | 100 | 100 |

TABLE 7
Effect of PVP complex and heparin on the glycoprotein C-deficient mutant
HSV-1 penetration into vero cells.

| | % Plaques | | | |
|---|---|---|---|---|
| | PVP | Heparin | Citrate buffer | No treatment |
| Time (min) | (100 μg/ml) | (1000 μg/ml) | (pH 3) | control |
| 0 | 6 | 62 | 0 | 89 |
| 10 | 15 | 90 | 0 | 91 |
| 20 | 17 | 100 | 0 | 100 |
| 30 | 24 | 100 | 0 | 100 |
| 40 | 48 | 100 | 18 | 100 |
| 50 | 91 | 84 | 28 | 100 |
| 60 | 100 | 100 | 100 | 100 |

### Example 18

### Identification of gC and gD as the PVP Complex Binding Target

Radiolabelling of HSV. Vero cell monolayers grown on tissue culture dishes (100×20 mm, Falcon #35-3003) was strayed of methionine by incubating for 1 h at 37° C. in MEM-Earle's methionine-free medium supplemented with 2% dialysed fetal calf serum. The monolayers were then chilled at 4° C. for 1 h, after which were infected with the HSV-1 at a multiplicity of infection of 5. The viral adsorption was allowed to occur at 4° C. for 1 h. The monolayers were then washed twice with methionine-free maintenance medium. Eight ml of the same medium containing 275 μCi of ³⁵S-methionine (NEN, specific activity 1175 Ci/mmol) was added to each plate. The cultures were labeled for 24 h, at which time the cells were detached with a rubber policeman and harvested by centrifugation (3,000×g, 10 min). The cell pellets were frozen at −20° C. Parallel monolayers that have not been infected with HSV were similarly radiolabeled and used as the mock-infected control. The cell pellets were lyzed with three freeze-thaw cycles and 1 ml of phosphate buffered saline containing 0.5% nonidet-P40 and deoxycholate (PBS-NP40-DOC) was added. After incubation on ice for 30 min and the lysate was used immediately in affinity chromatography.

Affinity chromatography. PVP-Sepharose beads were prepared by coupling PVP to CNBr-activated Sepharose (Pharmacia). The PVP-Sepharose column (2 ml bed volume) was equilibrated with 20 ml of PBS-NP40-DOC prior to use. HSV-1 infected or mock-infected Vero cell lysates in 1 ml of PBS-NP40-DOC was added to the column and incubated at room temperature for 5 min. The column was washed with 20 ml of PBS-NP40-DOC. Bound proteins were eluted with 10 ml of 5 mg/ml PVP complex in PBS-NP40-DOC. The proteins in the eluate were precipitated with 5% trichloroacetic acid (TCA), washed three times with cold acetone, and dissolved in 55 μl of Tris-SDS buffer (0.5 M Tris, pH 8, and 4% SDS).

Immuno-precipitation. The samples from affinity chromatography were boiled for 5 min and centrifuged (10,000×g, 5 min). A 5 μl aliquot was removed as total protein from PVP complex eluate. The remaining 50 μl sample was divided into two 25 μl aliquots. One ml of Triton X-100 buffer (50 mM Tris, pH 7.7, 0.15 M NaCl, 5 mM EDTA, and 1% Triton X-100) was then added to the sample. The rabbit polyclonal anti-gC (R46) and anti-gD (R7) antibodies were added to a final dilution of 1/40. The mixture was incubated at room temperature for 1 h with gentle rocking. Forty μl slurry of Protein A-agarose beads (Sigma Chemical Co., St. Louis, Mo.) was added. The mixture was further incubated at room temperature for 1 h. The Protein A beads were recovered by a 10 second centrifugation and washed 4× with 1 ml of bead wash buffer (50 mM Tris, pH 7.5, 150 mM NaCl, 5 mM EDTA). The beads were then boiled with 50 μl of SDS-PAGE buffer and the proteins were analyzed on a 7.5% SDS-PAGE gel (Laemmli, 1970) along with the high molecular weight protein markers (Bio-Rad Laboratories Ltd., Mississauga, ON). Following electrophoresis, the gel was fixed in 30% ethanol-10% acetic acid for 1 h. The gel was then washed with distilled water for 2×15 min and incubated with 1 M sodium salicyclic acid for 30 min. The gel was then drier with a gel dryer and autoradiographed on X-ray films (X-Omat, Kodak). The relative molecular masses of the protein bands were estimated from the Coomassie blue stained protein markers.

Identification of gC and gD as PVP binding targets. To identify the HSV-1 glycoproteins that interact with the PVP complex, the viral cultures were radiolabelled with ³⁵S-methionine, lysed with detergents and the solubilized proteins were subjected to affinity chromatography on a PVP-Sepharose column. The bound proteins were eluted with PVP complex and immuno-precipitated with anti-gC and and gD antibodies. The immuno-precipitate was recovered by protein A-agarose beads and analyzed by SDS-PAGE and autoradiography. As shown in FIG. 11A, two broad protein bands of 110-140 kDa and 48-65 kDa were present in the PVP complex eluate. Two of protein bands (110 kDa and 65 kDa) were isolated by imuno-precipitation with anti-gC antibody and a 60 kDa protein band was immunoprecipitated by the anti-gD antibody. These immuno-precipitated bands were absent from samples prepared from mock-infected Vero cell lysates (FIG. 11B).

### Example 19

### The in Vivo Anti-HSV-1 Activities of PVP Complex

This example is to determine the anti-HSV-1 efficacy of PVP complex creams in a guinea pig skin lesion model.

Material. Testing samples are PVP complex cream, topical use preparation, in brown color, 50 g/vial (Batch no. 200205), provided by the Department of Biology, the Chinese University of Hong Kong, Hong Kong. Positive drug control agent is 3% Acyclovir cream, white milky paste, 10 g/tube (Batch no. 991002, ZWYZZ (1996)-214602), provided by Wenzhou Pharmaceuticals of Zhejiang, China. Virus is Herpes simplex type 1 (HSV-1, Strain no. SM44), imported from The American Type Culture Collection. The virus strain was proliferated and preserved by the Department of Virological Diagnosis, Institution of Virology, Chinese Preventative Medical Academy. “Bunched needle” refers to seven individual needles (size No. 7) bunched together as one bunch and seven bunches were put together as one “Bunched needle”. Other related apparatuses: All other apparatuses and reagents were provided by the Department of Virological Diagnosis, Chinese Preventative Medical Academy.

Animals: Guinea pigs, half male and half female, 200-250 grams (animal license no. YDHZ SCXK11-00-0006), supplied by the Animal Center of the Chinese Medical Academy and Peking Union Medical College.

Toxicity evaluation of the testing samples. The zero toxic dose (TD₀) of testing PVP complex cream was 15 g % per animal for topical application (Data were provided by the Department of Biology, the Chinese University of Hong Kong, Hong Kong).

HSV-1 titer determination. The dorsal hairs of guinea pigs were removed with 8% barium sulfide (BaS) and the naked areas (40 cm²) were washed with 37° C. warm water and then dried with tissue paper. The naked dorsal skins were stabbed with a “bunched needle”, and the stabbed area was divided into 4 areas. The areas were infected with 4 concentrations of virus suspension (10⁻⁰, 10⁻¹, 10⁻² and 10⁻³ dilutions). Each diluted virus suspension was applied to 5 guinea pigs. Each skin area was infected with 30 μl of the diluted virus suspension. The animals were observed for 10 days for the development of lesions. The median infectious dose (ID₅₀) was then calculated on the basis of the infected areas with symptoms versus the entire infected dorsal skin area. The median infectious dose (ID₅₀) of HSV-1 (strain SM 44) to guinea pigs was determined to be 10^{-1.5}.

HSV-1 cutaneous lesion in guinea pig. The animals were grouped in 5. Animals within each group will be the same gender and have similar body weight. The hair was removed from dorsal side of the animal and the skin was abraded with needles as described above. The entire abraded area was infected with 150 μl of HSV-1 stock suspension.

Typical herpes lesions appeared on the infected area on the 4th day of infection. The extent of pathological changes was scored as described below. PVP complex, acyclovir, or base creams were then applied with cotton swabs on day 4 to the infected area. The amount of cream given to each animal was 1.5 gram per dose twice daily for a six-day duration.

Daily records of the pathological changes infected area were made according to the following criteria:

When the skin lesions developed up to ¼ of the total area, the pathological changes were scored as 1.0-1.6;

When the skin lesions developed on 2/4 of the total area, the pathological changes were scored as 1.7-2.4;

When the skin developed on ¾ of the total area, the pathological changes were scored as 2.5-3.2;

When the skin lesions developed 4/4 of the total areas, the pathological changes were scored as 3.3-4.0;

When the skin herpes scars developed up to ¼ of the total areas, the pathological changes were scored as 0.9-1.0;

When the skin herpes scars were up to 2/4 of the total areas, the pathological changes were scored as 0.7-0.8;

When the skin herpes scars were up to ¾ of the total areas, the pathological changes were scored as 0.5-0.6;

When the skin herpes scars were up to 4/4 of the total areas, the pathological changes were scored as 0.2-0.4;

When the skin herpes scars were all detached and lesions healed, the pathological changes were scored as 0.

Four days after infection, herpes lesions were observed on dorsal skins of the animals. The extent of pathological changes (EPC) was >3.8 out of 4.0 (Table 8). The animals were then given treatment twice daily for six days. A greater reduction in EPC was observed in animals that received PVP complex than the base cream group. In the 15% PVP group, a significant reduction in EPC was observed after 3 days of treatment as compared to the base cram. By day 11, the EPC was very small in the 15% PVP group. A similar pattern of EPC reduction was observed in the acyclovir control group. The inhibition rates calculated from the day 11 data for PVP cream treatment groups to pathological changes were 90%, 58%, 33% and 6%, respectively, while that of the acyclovir was 96%. The therapeutic effects of PVP complex and acyclovir was convincing when the data was compared to the virus control group in which the animals was infected but received no treatment. In this control group, typical herpes lesions appeared on the dorsal skin areas on the 4th to 6th days of viral infections with HSV-1. The extent of pathological changes was observed at an average value of 3.94 with blisters on most areas. In the 15% PVP group, the extent of lesions began to decrease on day 6 and scabs were appeared on the 4th day of drug application. All herpes lesions had formed scabs at the sixth day of drug application. In the virus control group, some animals showed the signs of paralysis in their lower limbs, indicating that the virus had invaded into the animal lower limb nerves with severe viral infections. The signs of paralysis were not observed in the PVP and acyclovir groups.

PVP complex cream was found to have significant in vivo anti-HSV-1 effects. Following a six-day treatment course, not only the morbidity was decreased, the HSV-1 infection course was also shortened. The potent therapeutic effects were comparable to acyclovir at the concentration tested. These results strongly suggest that PVP complex can be developed into an effective anti-HSV drug.

TABLE 8
Therapeutic effects of PVP complex creams on HSV-1 skin lesions in guinea pigs

| | | | | Extent of Skin Pathological Changes | | | | | | | | | |
|---|---|---|---|---|---|---|---|---|---|---|---|---|---|
| | | | | (Mean ± SD) and Inhibition rate | | | | | | | | | |
| | Agent | | Skin Area | Day 4* | | Day 5 | | Day 6 | | Day 7 | | Day 8 | |
| Testing | Dose | No. of | (width × | EPC | IR | EPC | IR | EPC | IR | EPC | IR | EPC | IR |
| Group | (g %) | Animals | length.) | (cm²) | (%) | (cm²) | (%) | (cm²) | (%) | (cm²) | (%) | (cm²) | (%) |
| PVP | 15 | 15 | 6 × 7 | 3.85 ± 0.15 | 0 | 3.78 ± 0.14 | 5 | 3.39 ± 0.10 | 14 | 2.74 ± 0.16 | 25 | 2.10 ± 0.26 | 36 |
| Cream | 10 | 15 | 6 × 7 | 3.83 ± 0.16 | 0 | 3.83 ± 0.14 | 3 | 3.57 ± 0.19 | 9 | 3.05 ± 0.43 | 16 | 2.57 ± 0.28 | 22 |
| | 5 | 15 | 6 × 7 | 3.84 ± 0.15 | 0 | 3.85 ± 0.11 | 3 | 3.75 ± 0.09 | 5 | 3.39 ± 0.17 | 7 | 3.13 ± 0.27 | 5 |
| | 2.5 | 15 | 6 × 7 | 3.81 ± 0.18 | 0 | 3.78 ± 0.16 | 5 | 3.71 ± 0.18 | 6 | 3.41 ± 0.20 | 6 | 3.15 ± 0.16 | 4 |
| | Base | 15 | 6 × 7 | 3.82 ± 0.02 | | 3.82 ± 0.02 | 3 | 3.99 ± 0.01 | 0 | 3.99 ± 0.01 | 0.02 | 3.58 ± 0.04 | 3 |
| | cream | | | | | | | | | | | | |
| Acyclovir | 3 | 15 | 6 × 7 | 3.87 ± 0.12 | 0 | 3.54 ± 0.22 | 11 | 2.77 ± 0.40 | 30 | 2.02 ± 0.30 | 44 | 1.21 ± 0.38 | 63 |
| Virus | | 15 | 6 × 7 | 3.84 ± 0.14 | | 3.96 ± 0.05 | | 3.94 ± 0.07 | | 3.63 ± 0.20 | | 3.29 ± 0.26 | |

| | Extent of Skin Pathological Changes | | | | | | | | |
|---|---|---|---|---|---|---|---|---|---|
| | (Mean ± SD) and Inhibition rate | | | | | | Statistical | | |
| | Day 9 | | Day 10 | | Day 11 | | Treatment | | |
| Testing | EPC | IR | EPC | IR | EPC | IR | T | P | Datum |
| Group | (cm²) | (%) | (cm²) | (%) | (cm²) | (%) | value | value | Analysis |
| PVP | 1.32 ± 0.36 | 54 | 0.69 ± 0.24 | 71 | 0.20 ± 0.17 | 90 | 12.25 | <0.01^{a} | Significant |
| Cream | | | | | | | | | therapeutic |
| | | | | | | | | | effects |
| | 1.87 ± 0.55 | 35 | 1.28 ± 0.53 | 47 | 0.86 ± 0.36 | 58 | 5.74 | <0.01 | Significant |
| | | | | | | | | | therapeutic |
| | | | | | | | | | effects |
| | 2.71 ± 0.36 | 6 | 2.17 ± 0.50 | 10 | 1.37 ± 0.26 | 33 | 3.83 | <0.01 | Therapeutic |
| | | | | | | | | | effects |
| | 2.85 ± 0.16 | 1 | 2.55 ± 0.30 | 0 | 1.93 ± 0.34 | 6 | 0.64 | >0.05 | No |
| | | | | | | | | | therapeutic |
| | | | | | | | | | effects |
| | 3.16 ± 0.01 | 4 | 2.66 ± 0.03 | 5 | 2.06 ± 0.03 | 7 | 1.8 | >0.05 | No |
| | | | | | | | | | therapeutic |
| | | | | | | | | | effects |
| Acyclovir | 0.82 ± 0.16 | 71 | 0.38 ± 0.18 | 84 | 0.09 ± 0.19 | 96 | 12.47 | <0.01 | Significant |
| | | | | | | | | | therapeutic |
| | | | | | | | | | effects |
| Virus | 2.87 ± 0.35 | | 2.40 ± 0.34 | | 2.06 ± 0.30 | | | | |
| Control | | | | | | | | | |

EPC: Extent of pathological changes;
IR: Inhibition rate = (Lesion area of virus control group − Lesion area of test group) / Lesion area of virus control group
*The typical herpes lesions on dorsal skins of guinea pigs developed to approximate 3.84 cm² at the 4th day of infection with HSV-1. Treatment was then given twice daily for six days. Observations continued to the eleventh day after drug treatment was suspended.
T values of statistical treatments were calculated from the 11th day data.
^{a}p value from Student's t test between the test group and the virus control group.

### Example 20

### The in Vivo Anti-HSV-2 Activities of PVP Complex

This example is provided to determine the therapeutic efficacy of PVP complex creams against herpes simplex virus 2 (HSV-2) infection in a mouse vaginal infection model.

Materials: Testing samples include 5%, 10% and 15% PVP complex creams in brown color, 50 g/vial. (Batch no. 200205), provided by the Department of Biology, the Chinese University of Hong Kong, Hong Kong. Positive drug control agent is 3% Acyclovir cream, white milky paste, 10 g/tube (Batch no. 991002, ZWYZZ (1996)-214602), provided by Wenzhou Pharmaceuticals of Zhejiang, China. Virus is Herpes simplex type II (HSV-2, Strain no. 333, batch no. 200104010), provided by the Department of Virological Diagnosis, Institution of Virology, Chinese Preventative Medical Academy. Needles for vaginal tract infection (size no. of 12) were prepared by the Department of Virological Diagnosis, Chinese Preventative Medical Academy. African green monkey kidney cells (Vero cells) were provided by the Department of Virological Diagnosis, Chinese Preventative Medical Academy. Physiological saline and cell culture medium was provided by the Department of Virological Diagnosis, Chinese Preventative Medical Academy. Inverted microscope was provided by the Department of Virological Diagnosis, Chinese Preventative Medical Academy. Carbon dioxide incubator was provided by the Department of Virological Diagnosis, Chinese Preventative Medical Academy. And other related apparatuses: provided by the Department of Virological Diagnosis, Chinese Preventative Medical Academy.

Animals: BALB/c mice, female, body weight range of 18-20 grams. Animal license no. YDHZ 01-3003, supplied by the Animal Center of the Chinese Medical Academy and Peking Union Medical College.

Toxicity evaluation of the PVP. The zero toxic dose (TD₀) of testing PVP complex cream was 15 g % per mouse for topical application (Data were provided by the Department of Biology, the Chinese University of Hong Kong, Hong Kong).

Virus titer determination. HSV-2 stock was diluted from 10⁻⁰ to 10⁻³. The diluted virus suspension (0.03 ml) was injected into the vagina by inserting the needle in a rotatory manner with a stabbing motion along the vaginal wall. Each dilution of virus was inoculated to 10 mice per group. The mice were kept under observation for 12 days, by which time the animals developed vaginitis or lethality occurred. The median lethal dose (LD₅₀) was then calculated. With a parallel group, vaginal samples were taken at different time of viral infection. The virus titers (median tissue culture-infective dose, TCID₅₀) of the samples collected at the respective infection stages were determined by culturing on Vero cell.

In Vivo Anti-HSV-2 Activities of PVP Complex Creams:

1. Target of the experiments: The mice could develop viral vaginitis following infection with HSV-2. After treatment with the PVP complex creams, the morbidities, mortalities, average surviving days, protection rates, and life span extension rates were calculated. Statistical analysis, t values and p values, was calculated in comparison with the virus control group. Efficacy of the testing samples will be evaluated on the basis of statistical calculation. The median lethal dose (LD₅₀) of HSV-2 stock was determined to be 10^{-2.35}. The virus titer (50% tissue culture-infective dose, TCID₅₀) of the vaginal tract samples after viral infection was determined to be 10^{-2.35} by Vero cell culture.

2. Infection with virus: The animals were infected with a virus dose of 10 LD₅₀ in using the method as described above. The BALB/c mice were divided into 3 test groups (5, 10, and 15% PVP complex), one positive control (acyclovir), one negative control group (base cream), and one no-treatment (virus control) group. The number of animals per group was 10. Ten additional animals that were not infected with HSV-2 and received no treatment served as the uninfected control group.

Symptoms of viral vaginitis were observed on the third day of infection. The symptoms were topical edema of vaginal tracts with turbid secretions. Treatment began on day 3 post-infection. This was achieved by applying the cream to the vaginal tract with cotton swabs. The creams were given at a dose of 2 mg per mouse twice daily for a six-day duration. Mortality and the number of days for lethality to occur were recorded. The experiment was repeated twice. The protection rates and life span extension rates were calculated. The efficacy of testing agents were judged in comparison with the positive control group on the basis of the calculated t values and p values.

Viral vaginitis was observed on the third day following HSV-2 infection. The survival rate and number of days for lethality to occur were recorded. The latter was expressed as the average life span in days.

TABLE 9
Anti-HSV-2 efficacy of PVP complex creams in a mouse genital infection model.

| | | | | | | | Median | | Median |
|---|---|---|---|---|---|---|---|---|---|
| | No. of | | | | Average | | Effective | Life Span | Effective |
| Agent Dose | animals | No. of | No. of | Survival | Life Span | Protection | Dose | Extension | Dose |
| (g %) | (n) | survival | death | (%)^{b} | (Day)^{c} | Rate (%)^{d} | (ED₅₀) | Rate (%)^{c} | (ED₅₀) |
| PVP Cream | | | | | | | | | |
| 15 | 30 | 22 | 8 | 73.3 | 11.2 ± 1.33 | 71.4 | 6.62 | 75 | 5.32 |
| | | | | | | (p < 0.01)^{f} | | (p < 0.01) | |
| 10 | 30 | 19 | 11 | 63.3 | 10.7 ± 1.82 | 60.7 | | 67.2 | |
| | | | | | | (p < 0.01) | | (p < 0.01) | |
| 5 | 30 | 14 | 16 | 46.7 | 9.5 ± 2.50 | 42.9 | | 48.4 | |
| | | | | | | (p < 0.01) | | (p < 0.01) | |
| 3% | 30 | 24 | 6 | 80 | 11.6 ± 0.96 | 78.6 | | 81.2 | |
| Acyclovir | | | | | | (p < 0.01) | | (p < 0.01) | |
| Base cream | 30 | 4 | 26 | 13.3 | 7.2 ± 2.07 | 7.1 | | 12.5 | |
| | | | | | | (p > 0.05) | | (p > 0.05) | |
| Uninfected | 30 | 30 | 0 | 100 | | | | | |
| Control | | | | | | | | | |
| Virus | 30 | 2 | 28 | 6.7 | 6.4 ± 1.73 | | | | |
| Control^{a} | | | | | | | | | |

^{a}Animals infected with the virus but received no treatment.
^{b}Survival = (Number of survival / Number of animals) × 100.
^{c}Average life span of animals that died.
^{d}Protection rate = (Survival of virus control group − Survival of test group) / Survival of virus control group.
^{e}Life sapn extension rate = (Average life span of test group − Average life span of virus control group) / Average life span of virus control group
^{f}p value from Student' t test between the test group and the virus control group.

As shown in Table 9, the animals that received the PVP complex cream showed a dose-dependent survival rate with the highest survival at the 15% concentration. The survival rate for the 15% PVP complex group is comparable to that of 3% acyclovir. Animals received the base cream showed a very low survival rate. The average life span of the animals that died from the infection was highest for the 15% PVP complex and the acyclovir groups. The protection rates of the three concentrations of PVP complex were calculated to be about 71.4%, 60.7% and 42.9%, respectively. The median effective dose (ED₅₀) for protection was about 6.62%. The life span extension rates were calculated to be about 75%, 67.2% and 48.4%, respectively. The median effective dose (ED₅₀) for life span extension was about 5.32%. The protection rate of 3% acyclovir was 78.6%, and the life span extension rate was 81.3%. The results showed that PVP complex has an excellent therapeutic effects against HSV-2 infection in the mouse model.

3. Recovery of HSV-2 from infected animals. The animals were infected as above. Symptoms of viral vaginitis in the infected mice were observed on the third day infection. Before treatment, vaginal tract samples were collected with cotton swabs and transferred to 0.5 ml of physiological saline and stored at −25° C. The animals were treated with the creams as above for 6 days. One day following the completion of the treatment, vaginal samples were obtained and were kept at −25° C. Samples were also obtained from dead animals. In a parallel group, vaginal samples were taken at different times of viral infections. The vaginal samples were diluted by ⅕ in cell culture medium and used to infect Vero cells.

The presence of HSV-2 on the vaginal samples was determined by cytopathic effect (CPE) to Vero cells. The extent of CPE shown in each sample will be observed and recorded. “+”=up to 25% cells showing CPE; “++”=26˜50% cells showing CPE; “+++”=51˜75% cells showing CPE; “++++”=76˜100% cells showing CPE. The experiment was repeated twice.

The effect of PVP complex treatment on the clearance of HSV-2 was assessed from vaginal sampling before and after treatment. The results are shown in Table 10.

TABLE 10
Therapeutic effects of PVP complex treatment on the reduction of HSV-2 in the genital tract.

| | | No. of | No. of samples | | | Statistical | | |
|---|---|---|---|---|---|---|---|---|
| | | vaginal | containing HSV-2 | | | Calculation | | |
| | | samples | Before | After | Therapeutic | (After Treatment) | | |
| Agent Dose (g %) | | (1/mouse) | Treatment | Treatment | Rate (%)^{a} | X² | p Value | Data Analysis |
| PVP Cream | 15 | 30 | 30 | 9 | 68 | 23.3 | p < 0.01^{b} | Significant |
| | | | | | | | | antiviral effects |
| | 10 | 30 | 30 | 16 | 42 | 12.3 | p < 0.01 | Antiviral effects |
| | 5 | 30 | 30 | 21 | 25 | 5.3 | p < 0.01 | Antiviral effects |
| Acyclovir | 3 | 30 | 30 | 8 | 71 | 27.8 | p < 0.01 | Significant |
| | | | | | | | | antiviral effects |

| | | | | | | | |
|---|---|---|---|---|---|---|---|
| Base cream | 30 | 0 | 27 | 4 | 0.22 | p > 0.05 | No antiviral effects |
| Uninfected | 30 | 0 | 0 | | | | |
| Control | | | | | | | |
| Virus Control | 30 | 30 | 28 | | | | |

^{a}Therapeutic rate = (Percent of HSV-2 positive samples in virus control group − Percent of HSV-2 positive samples in test group) / Percent of HSV-2 positive samples in virus control group HSV-2 after treatment
^{b}p value from Student's t test between the test group and the virus control group.

The animals that received 15, 10 and 5% PVP complex showed therapeutic rates of about 68, 42 and 25%, respectively. The highest rate was comparable to that of animals received 3% acyclovir. These results indicate that PVP complex treatment reduced the HSV-2 load in the genital tract and correlate well with the survival data reported in Table 9.

On the basis of experimental results, it was concluded that 5% and higher concentrations of PVP complex creams have in vivo anti-HSV-2 therapeutic effects.

### Example 21

### In Vivo Toxicity Testing of Anti-Herpes Compound(s) from Prunella vulgaris

The in vivo toxicity of invention compound can be tested using means known in the art, for example, a two-step procedure on albino mice as follows:

Step 1: Dose Ranging Determination.

To determine the dose which will produce a toxic effect in mice, the anti-herpes extract PVP dissolved in 0.4 ml of distilled water was administered orally, via a feeding tube, to BALB/c female mice (8 month-old, 22 to 23 gram). A low dose (e.g., 25 mg/kg) of the new compound is administered orally to one animal which is then observed hourly for 24 hours and thereafter, every eight hours, with continuous monitoring daily for 14 days. A second animal receives double the dose of the first animal. The process is then repeated for subsequent animals, each receiving a dose twice that of the previous animal to a maximum of 2000 mg/kg (OCED guidelines 1995). A total of ten animals are used to establish dosing in this step. The amount of PVP administered per animal was 25, 100, 200 400 and 800 mg/kg body weight.

At each of the doses, the animals survived and showed no signs of in a moribund state. A moribund state is characterized by symptoms such as shallow, labored or irregular respiration, muscular weakness or tremors, absence of voluntary response to external stimuli, inability to remain upright, cyanosis and coma. Any one of these indicators will mark a moribund condition, and the animal is euthanized immediately. However, if no moribund effects or lethality were observed, the animals are killed and selected organs (heart, lung, liver, spleen, kidney, brain, muscle, uterus) sampled for histopathology at day 14. Since no moribund effects or lethality was observed, the animals were euthanized and the organs of each animal were examined. Histological examination of the selected organs showed no inflammation or any other pathological signs. These results indicate that PVP has no in vivo toxicity at a concentration up to 800 mg/kg.

Step 2: Approximate Acute Toxicity (LD₅₀) Determination.

Using dose ranging determinations from Step 1, three appropriate dose levels are established for LD₅₀ determination. Ten animals are used for each dose level, and monitored hourly for 24 hours and thereafter, every eight hours, with continuous monitoring daily for 14 days. Animals exhibiting moribund signs are killed immediately; all animals are killed at 14 days and their organs sampled for histology. Using the data from these experiments, actual LD₅₀ is estimated by extrapolation and mathematical manipulations using known methods in the art (see, for example DePass. in Toxicology Letters 49:159 (1989).

Compounds present in the untreated extract (see Example 3) and in the purified fraction (Example 5) are active in suppressing the cytopathogenic effects of HSV in vitro. Based on this latter activity and certain of their biochemical characteristics (particularly their anionic properties) these preparations of the extract are capable of suppressing the cytopathogenicity of other enveloped viruses. This extrapolation is supported by Baba et al (see Antimicrobial Agents and Chemotherapy 32: 1742 (1988)).

In conclusion, it has been shown that the invention compositions, obtained, for example, from the spikes of Prunella vulgaris, are effective agents for the treatment of the cytopathogenic effects of an enveloped virus in mammals. These results provide insight into the pathway of action of the purified extract fraction within enveloped viruses.

While the invention has been described in detail with reference to certain preferred embodiments thereof, it will be understood that modifications and variations are within the spirit and scope of that which is described and claimed.

What is claimed is:

1. A composition comprising a lignin-carbohydrate complex, wherein said complex inhibits viral infection of mammals.

2. The composition of claim 1, wherein the ratio of lignin to carbohydrate in said complex is about 2:1.

3. The composition of claim 1, wherein said lignin comprises oxidized derivatives of vanillin, syringaldehyde and p-hydroxybenzaldehyde.

4. The composition of claim 1, wherein said carbohydrate is characterized as a water soluble polyanionic polysaccharide comprising glucose, galactose, xylose, arabinose, rhamnose, mannose, and galacturonic acid,
wherein glucose is a major constituent as analyzed by paper chromatography;
wherein said carbohydrate comprises 31-35% carbon, 3-4% hydrogen, 0.5-1% nitrogen or 2-3% sulfur;
wherein about 42% carbohydrate is expressed as glucuronic acid; and
where about 7.5% uronic acid is expressed as glucuronic acid.

5. The composition of claim 1, wherein said composition has a molecular weight of about 8.5 kDa;
wherein said composition has little or no anti-coagulant activity as measured by the prothrombin time test, and
wherein a therapeutically effective amount of said composition has little or no in vivo toxicity to a mammalian subject to which it is administered.

6. The composition of claim 5, wherein said composition is stable to temperatures in the range of about 95-100° C. for 4 hours;
has a pH of 5.5. when dispersed in an aqueous solution at a concentration of about 1 mg/ml; and
is substantially insoluble in methanol, ethanol, butanol, acetone and chloroform.

7. The composition of claim 6, wherein said composition is non-proteinaceous having a retention time of 3.56 min when subjected to reverse-phase high pressure liquid chromatography (HPLC) on a C18 column (25 cm×4.6 mm ID, 5 m, Supelcosil LC-18, Sigma) and eluted with a mixture of 5% water and 95% acetonitrile at a flow rate of 0.3 ml/min;
binds to Alcian blue and to DEAE Sepharose at neutral pH; and
has a strong UV absorption peak at 202 nm with a shoulder at 280 nm extending to 380 nm when dispersed in distilled water.

8. The composition of claim 1, wherein said composition has anti-cytopathogenic effects for an enveloped virus, and wherein said composition inhibits viral infection both before virus binding and after virus binding and penetration.

9. The composition of claim 8, wherein said anti-cytopathogenic effects are anti-HSV activities.

10. The composition of claim 9, wherein said anti-HSV activities are anti-HSV-1 activities.

11. The composition of claim 9, wherein said composition has a direct inhibition effect to HSV-1.

12. The composition of claim 9, wherein said composition blocks the penetration of HSV-1 into Vero cells.

13. The composition of claim 9, wherein said anti-HSV activities are anti-HSV-2 activities.

14. The composition of claim 1, wherein said composition is derived from the Napetoideae subfamily of plants.

15. The composition of claim 1, wherein said composition is derived from cells of the plant Prunella vulgaris.

16. The composition of claim 1, wherein said composition is extracted and purified from the spikes of the plant Prunella vulgaris.

17. A pharmaceutical formulation comprising a composition of claim 1 and a pharmaceutically acceptable carrier therefor.

18. A composition comprising a lignin-carbohydrate complex, wherein a ratio of lignin to carbohydrate is about 2:1,
wherein said complex has molecular weight of about 8.5 kDa;
wherein said lignin comprises oxidized derivatives of vanillin, syringaldehyde and p-hydroxybenzaldehyde;
wherein said carbohydrate is characterized as a water soluble polyanionic polysaccharide comprising glucose, galactose, xylose, arabinose, rhamnose, mannose, and galacturonic acid, wherein glucose is a major constituent as analyzed by paper chromatography;
wherein said complex is effective for treatment of the cytopatogenic effects of an enveloped virus in a mammal; and
wherein a therapeutically effective amount of said composition has little or no in vivo toxicity to a mammalian subject to which it is administered.

19. A method for producing a composition according to claim 1, said method comprising:
(a) extracting spikes of Prunella vulgaris;
(b) precipitating and purifying the extract obtained from (a);
(c) fractionating purified extract of (b); and
(d) analyzing purified fraction of (c) for antiviral activities.

20. A method for the treatment of viral infection in a mammal comprising administering to the mammal an effective amount of a composition of claim 17.
